(19) United States
(12) Patent Application Publication
(10) Pub. No.: US 2020/0309767 A1
(43) Pub. Date: Oct. 1, 2020

(54) HIGH-THROUGHPUT IMAGING-BASED METHODS FOR PREDICTING CELL-TYPE-SPECIFIC TOXICITY OF XENOBIOTICS WITH DIVERSE CHEMICAL STRUCTURES

(71) Applicant: AGENCY FOR SCIENCE, TECHNOLOGY AND RESEARCH, Singapore (SG)

(72) Inventors: Lit-Hsin LOO, Singapore (SG); Jia Ying LEE, Singapore (SG); Ran SU, Singapore (SG); Daniele ZINK, Singapore (SG); Sijing XIONG, Singapore (SG)

(21) Appl. No.: 15/777,653

(22) PCT Filed: Nov. 9, 2016
(86) PCT No.: PCT/SG2016/050554

§ 371 Date: May 18, 2018

(30) Foreign Application Priority Data
Nov. 23, 2015 (SG) 10201509598X

(51) Int. Cl. G01N 33/50; G01N 1/30; G06T 7/00; G06T 7/10; G01N 21/64

(52) U.S. Cl. CPC G01N 33/5014; G01N 1/30; G06T 7/0012; G06T 7/10; G01N 2021/6439; G01N 2001/302; G06T 2207/10056; G06T 2207/10064; G06T 2207/30024; G01N 21/6428

(57) ABSTRACT

The present invention provides methods for the prediction of in vivo cell-specific toxicity of a compound that combines high-throughput imaging of cultured cells, quantitative phenotypic profiling, and machine learning methods. More particularly, the invention provides a method for the prediction of in vivo renal proximal tubular-, bronchial-epithelial-, and alveolar-cell-specific toxicities of a soluble or particulate compound that comprises contacting cultured human kidney and pulmonary cells with the compound at a range of concentrations, then labeling the cells with DNA, γH2AX and actin markers and obtaining textural features, spatial correlation features, ratios of the markers, intensity features, cell count and morphology, estimating dose response curves and performing automatic classification of the compound using a random-forest algorithm.

### FIELD OF THE INVENTION

The present invention provides methods for the prediction of in vivo cell-specific toxicity of a compound that combines high-throughput imaging of cultured cells. More particularly, the invention provides a method for the prediction of in vivo renal proximal-tubular-, bronchial-epithelial-, and alveolar-cell-specific toxicities of a soluble or particulate compound that combines high-throughput imaging of cultured human kidney and pulmonary cells.

### BACKGROUD OF THE INVENTION

The kidney and lung play an important role in the metabolism and/or elimination of xenobiotics from the plasma. Foreign compounds originating from medicine, food, or the environment are transported and metabolized by the renal proximal tubular cells (PTCs), bronchial epithelial cells (BECs), and alveolar cells (AVCs). After uptake, xenobiotics and their metabolites/intermediates may damage the PTCs, BECs, and AVCs; and lead to acute kidney/lung injuries or chronic kidney/lung diseases. Therefore, accurate methods for predicting PTC-, BEC-, and AVC-specific toxicities are critical for the safety assessment of xenobiotics, and the management of the health and environmental hazards posed by these compounds.

There are several existing approaches for predicting xenobiotic toxicity in human. Animal testing is a standard approach, but suffers from the problems of long turnaround time, low throughput, and sometimes poor prediction of human toxicity (Krewski et al., J Toxicol Environ Health Part B 13: 51-138 (2010)). This approach is especially unsuitable for evaluating the large numbers of existing and ever-increasing numbers of novel synthetic compounds, such as chemicals and nanoparticles. In fact, the current interest in alternatives to animal testing is driven by the requirement for efficient testing of large numbers of compounds with diverse chemical structures and injury mechanisms. This is, for instance, reflected by current legislations, such as the regulation on “Registration, Evaluation, Authorization and restriction of Chemicals” (REACH) in the European Union (Lilienblum et al., Arch Toxicol 82: 211-236 (2008)). Computational modeling of quantitative structure-activity relationships (QSAR) is a rapid approach and works well for compounds with specific or well-understood chemical structures or mechanisms (Cherkasov et al., J Med Chem 57: 4977-5010 (2014)). However, most QSAR models do not consider the biological contexts of compound exposure, and therefore have limited applications in predicting the complex biological responses, such as organ-specific toxicity, of compounds with diverse chemical structures. Finally, in vitro assays based on immortalized, primary, or stem-cell-derived human cells may provide a balance between throughput and physiological relevance.

Most of the existing cell-based nephrotoxicity assays are based on either cell death/health endpoints (Lin and Will Toxicol Sci 126: 114-127 (2012); Jang et al., Integr Biol 5: 1119-1129 (2013)) or gene expression markers (Hoffmann et al., Toxicol Sci 116: 8-22 (2010)). However, most of the current cell-based assays were either tested with very small numbers of nephrotoxicants (usually <5) (Jang et al., Integr Biol 5: 1119-1129 (2013); Tiong et al., Mol Pharm 11: 1933-1948 (2014)), or were poorly predictive of organ-specific toxicity in large-scale studies (Lin and Will Toxicol Sci 126: 114-127 (2012)). Therefore, accurate prediction of nephrotoxicity remains challenging, and there is currently no regulatory approved in vitro test for nephrotoxicity. Recently, we have developed nephrotoxicity models based on compound-induced interleukin (IL)-6/8 expression levels in immortalized and primary human PTCs (Li et al., Toxicol Res 2: 352-365 (2013); Su et al. 2014), human embryonic stem cell—(Li et al., Mol Pharm 11: 1982-1990 (2014)), and iPSC-derived PTC-like cells (Kandasamy et al., Sci Rep. doi: 10.1038/srep12337 (2015)). We rigorously evaluated the performance of these models using a large set (˜30-40) of structurally diverse compounds, which included non-PTC-toxic nephrotoxicants and non-nephrotoxic compounds as negative reference compounds. Due to the relatively high test accuracies (˜75.3%) of these models, we hypothesize that there may be PTC-specific injuries that are commonly induced by PTC toxicants with diverse structures and targets. Furthermore, the RNA isolation and qPCR steps of the IL-6/8 measurements used in our previous studies are difficult and costly to be automated for high-throughput applications. Therefore, there is still a need to develop an alternative high-throughput, cost-effective, and accurate nephrotoxicity prediction approach, which may also provide new insights into the cell injuries and responses induced by these compounds.

Similarly, several groups have tried to build in vitro pulmonary toxicity models based on immortalized cell lines and primary cells (Seagrave et al., Exp Toxicol Pathol 57: 233-238 (2005); Sayes et al., Toxicol Sci 97(1): 163-180 (2007)). Most of these models used lactate dehydrogenase (LDH) release or inhibition of 3-(4,5-dimethylthiazol-2-yl)2,5-diphenyl-tetrazolium bromide (MTT) reduction as toxicity endpoints. However, they have very poor prediction of in vivo toxicity even for moderate numbers of pulmonary toxic compounds (˜5-10) (Seagrave et al., Exp Toxicol Pathol 57: 233-238 (2005); Sayes et al., Toxicol Sci 97(1): 163-180 (2007)). To the best of our knowledge, there is currently no in vitro pulmonary toxicity model that can accurately predict the in vivo toxicity of large numbers of (>20) compounds.

Xenobiotic-induced injuries impair cellular functions and lead to changes in cellular phenotypes, such as reorganization and changes in cellular and subcellular structures. One of the main advantages of predicting toxicity based on cellular phenotypes is that the cell injury mechanisms do not need to be defined a priori. This is especially useful for building models for a diverse set of xenobiotic compounds that may induce the same types of injury and responses, but through different biochemical mechanisms. Models based on specific mechanisms may only cover specific classes of compounds, and not be generally applicable to other compounds (Tiong et al., Mol Pharm 11: 1933-1948 (2014)). Several previous studies (O'Brien et al., Arch Toxicol 80: 580-604 (2006); Abraham et al. J Biomol Screen 13: 527-537 (2008); Xu et al., Toxicol Sci 105: 97-105 (2008); Tolosa et al., Toxicol Sci 127: 187-198 (2012)) and patents (Hong and Ghosh, U.S. patent application Ser. No. 14/334,453 (2015)) have used imaging to measure changes in cellular phenotypes and predict the toxicity of xenobiotic compounds. There are two key limitations of these previous imaging-based toxicity assays. First, most of them only extract spatial-independent features from the cellular images. Two of the most commonly used spatial-independent features are the sum of the intensity values of all the pixels in a cellular or subcellular region, and the area of a cellular or subcellular region (O'Brien et al., Arch Toxicol 80: 580-604 (2006); Abraham et al. J Biomol Screen 13: 527-537 (2008); Xu et al., Toxicol Sci 105: 97-105 (2008); Tolosa et al., Toxicol Sci 127: 187-198 (2012); Hong and Ghosh, U.S. patent application Ser. No. 14/334,453 (2015)). These features do not consider the locations of individual pixels, and will give exactly the same values even if the positions of the underlying pixels are randomly shuffled. Second, most of these assays are only based on half maximal inhibitory concentration (IC₅₀), minimum effective concentration (MEC), or other concentration-based parameters of the dose response curves of the extracted features or cell-death readouts (O'Brien et al., Arch Toxicol 80: 580-604 (2006); Abraham et al. J Biomol Screen 13: 527-537 (2008); Tolosa et al., Toxicol Sci 127: 187-198 (2012)). Due to these two limitations, most of these previous works either did not evaluate or obtained very poor performances in predicting organ-specific toxicity. For example, the imaging-based assay developed by O'Brien et al. misclassified 45 of the 50 tested compounds that are non-toxic to liver, but toxic to other organs, as liver toxic (specificity=10% only).

### SUMMARY OF THE INVENTION

The present invention provides an alternative high-throughput, cost-effective, and accurate cell-type-specific toxicity prediction approach.

In a first aspect of the invention there is provided an in vitro method of predicting whether a test compound will be toxic for a specific cell type in vivo, the method comprising:
(a) contacting at least one test population of cells with the test compound at a single concentration or over a range of concentrations,
(b) labeling and imaging the cells with one or more biomolecular markers,
(c) segmenting the cells and identifying whole-cell regions and one or more subcellular regions from the cells,
(d) determining if cell loss or death has occurred at the highest test concentrations (if so, stop and predict the compound is toxic),
(e) obtaining one or more quantified spatial-dependent and -independent phenotypic features in the test populations,
(f) obtaining multiple dose response curves (DRCs) from the features,
(g) obtaining quantified parameters of the DRCs, and
(h) comparing the quantitated DRC parameters to a reference set of quantitated DRC parameter data; said reference quantitated DRC parameter data being derived from two groups;
   (i) compounds with known in vivo toxicity to the cell type, and (ii) compounds not known to be toxic to the cell type in vivo.

In a preferred embodiment of the invention, the method comprises:
(a) contacting multiple test populations of cells;
(b) labeling and imaging the cells with one or more biomolecular markers,
(c) segmenting the cells and identifying whole-cell regions and one or more subcellular regions from the cells,
(d) determining if cell loss or death has occurred at the highest test concentrations (if so, stop and predict the compound is toxic),
(e) obtaining one or more quantified spatial-dependent and -independent phenotypic features in the test populations,
(f) obtaining multiple dose response curves (DRCs) from the features,
(g) obtaining quantified parameters of the DRCs, and
(h) comparing the quantitated DRC parameters to a reference set of quantitated DRC parameter data; said reference quantitated DRC parameter data being derived from two groups;
   (i) compounds with known in vivo toxicity to the cell type, and (ii) compounds not known to be toxic to the cell type in vivo.

In a preferred embodiment of the method of the invention, said specific cell type is selected from the group comprising renal proximal tubular cells (PTCs), bronchial epithelial cells (BECs), and/or alveolar cells (AVCs).

In a preferred embodiment of the method of the invention, step (h) comprises comparing the quantitated DRC parameters to a reference set of quantitated DRC parameter data; said reference quantitated DRC parameter data being derived from two groups;
in the case of PTCs; (i) compounds with known in vivo PTC toxicity, and (ii) compounds nephrotoxic but not known to be PTC toxic in vivo and compounds not known to be nephrotoxic in vivo; or
in the case of BECs or AVCs; (i) compounds with known in vivo BEC or AVC toxicity, and (ii) compounds pulmonotoxic but not known to be BEC or AVC toxic in vivo and compounds not known to be pulmonotoxic in vivo.

Preferred embodiments of the invention propose a method for the prediction of in vivo cell-specific toxicities utilizing measurements of spatial-dependent chromosomal and cytoskeletal features of the cells, their maximal response values, and a cascade automated classification algorithm.

In a preferred embodiment of the method of the invention, said one or more quantitated phenotypic features are associated with characteristics selected from the group comprising DNA damage response, actin filament integrity, whole-cell morphology, and cell count.

In another preferred embodiment of the method of the invention, said one or more phenotypic features are quantitated based on (i) one or more of the spatial-dependent features selected from the group comprising textural features, spatial correlation features, and ratios of markers at different subcellular regions; and (ii) one or more of the spatial-dependent features selected from the group comprising intensity features, cell count and morphology.

In another preferred embodiment of the method of the invention, the cell markers are selected from the group comprising, DNA, actin and the DNA damage response marker histone H2AX phosphorylated on Serine 139 (γH2AX)

In another preferred embodiment of the method of the invention, the DRC parameters are quantitated using the maximum response value Δₘₐₓ of the DRC of the test compound for each phenotypic feature.

In another preferred embodiment of the method of the invention, the said one or more phenotypic features consist of the total actin intensity level at the inner cytoplasmic region; mean angular second moment (ASM) of DNA GLCM at the nuclear region; standard deviation of the information measure of correlation 2 of γH2AX GLCM at the whole-cell region; and cell count.

In another preferred embodiment of the method of the invention, nephrotoxicity is predicted using a random-forest algorithm.

In a second aspect of the invention there is provided a computer-implemented method of predicting in vivo cell toxicity of a test compound using a test population of the cells subjected to the test compound in vitro, the method comprising:
(a) receiving, by a computer processor, an image of the test population of the cells;
(b) extracting, by the computer processor, one or more spatial-dependent phenotypic features associated with the test population of cells from the image, the one or more spatial-dependent phenotypic feature characterizing a spatial distribution of biomolecules associated with the cells;
(c) obtaining one or more quantitated dose response curve (DRC) parameters describing the DRCs of the respective one or more spatial-dependent phenotypic features; and
(d) inputting said one or more quantitated DRC parameters to a predictive model to generate a prediction of in vivo cell toxicity of the test compound.

In another preferred embodiment of the method of the invention, said image comprises a plurality of images each representing the test population of cells imaged using a respective imaging channel emphasizing a type of biomolecule associated with the cells. For example, the respective imaging channel is for imaging a type of fluorescent markers targeting a specific type of biological composition or molecules within the cells.

In another preferred embodiment of the method of the invention, each of the plurality of images represents a distribution of a type of biomarker targeting the corresponding type of biomolecule.

In another preferred embodiment of the method of the invention, step (b) comprises segmenting the cells using the image, and extracting the one or more spatial-dependent phenotypic features using intensity values of the image corresponding to the segmented cells.

In a preferred embodiment of the method of the invention, the one or more spatial-dependent phenotypic features are selected from the group comprising features characterizing DNA structure alterations, chromatin structure alterations and Actin filament structure alterations of the cells.

In a preferred embodiment of the method of the invention, step (d) comprises classifying the test compound to either toxic or non-toxic for the cells.

In a preferred embodiment of the method of the invention, the predicative model is obtained using a supervised learning algorithm trained with a set of training data.

Another aspect of the invention provides a computer system having a computer processor and a data storage device, the data storage device storing non-transitory instructions operative by the processor to perform a computer-implemented method according to any aspect of the invention.

Another aspect of the invention provides a non-transitory computer-readable medium, the computer-readable medium having stored thereon program instructions for causing at least one processor to perform a computer-implemented method according to any aspect of the invention.

### BRIEF DESCRIPTION OF THE FIGURES

FIG. 1. Shows reference xenobiotic compounds have diverse chemical structures. a) Categorization of the reference xenobiotic compounds according to their sources or applications. b) Multi-dimensional scaling plot showing the chemical structure dissimilarity based on Tanimoto coefficients between all the reference compounds (MDS1/2=the first and second coordinates of the multi-dimensional scaling, dashed line=a cluster of compounds with simple and similar chemical structures. All industrial chemicals are grouped into this cluster together with other compounds irrespective of their known PTC toxicity. Many compounds within the cluster are on top of each other.

FIG. 2. Shows an overview of the image and data analysis procedures.

FIG. 3. Shows quantitative image-based phenotypic profiles of primary human proximal tubule cells treated with the reference compounds. a) Immunofluorescence images showing the four fluorescence markers used in the HPTC-A dataset for primary PTCs treated with the DMSO control or 500 μg/mL cisplatin (scale bar=20 μm). b) Single-cell probability distribution functions for the raw coefficient of variation (CV) of actin intensity values measured from primary PTCs treated with different concentrations of citrinin or DMSO. Exemplary fluorescent images for the actin stains are shown above the distribution function plots (scale bar=20 μm). c) Dose response curves (DRC) for changes in the CV of actin intensity induced by three of the reference compounds (ochratoxin A and citrinin are PTC-toxic compounds, ribavirin is a non-PTC-toxic compound). The maximum response value (Δₘₐₓ) for each compound was determined from its response curve at 5 mM. d) Heat map showing the Δₘₐₓ values for all the 129 phenotypic features (rows) measured from primary human PTCs treated with 44 reference compounds (columns). (Dendrograms=hierarchical clustering of the compounds or features based on the Δₘₐₓ values, dash line=separation between the two major clusters identified from the clustering of compounds).

FIG. 4. Shows automated cell segmentation. An example of a full-frame immunofluorescence image showing automatically identified cell boundaries (white lines) and nuclear boundaries (grey lines) of primary human proximal tubule cells. Cells that touched the image boundary were not included in our analysis.

FIG. 5. Shows human in vivo nephrotoxicity prediction based on in vitro DNA and cytoskeleton features of PTCs. a) Schematic showing the procedure for identifying the best single feature (f_{best}) from all the 129 phenotypic features. The test balanced accuracies of the classifiers based on the best single features from different b) feature marker groups or c) feature type groups in the HPTC-A dataset. d) Schematic showing the procedure for identifying the best feature subset (Fₛ) from all the 129 phenotypic features. e) An example of the output of automated feature elimination from one of the 10 cross validation folds. The performance of the feature subset selected during each iteration of our recursive feature elimination algorithm is shown, starting from all features to the last retained feature (gray dots=test balance accuracy of the feature subset retained during each iteration, black line=spline interpolation of all the test balance accuracy values, black dot=local maximum with the smallest number of features, horizontal dashed lines=upper and lower 5-percentiles or limits of the Gaussian distribution centered around the last local maximum, white dot=the test balanced accuracy of the final selected feature subset, which is the subset with the smallest number of features and accuracy value between the upper and lower limits). In this example, the final selected number of features was four (vertical dashed line). Any further elimination of features would reduce the performance of the classifier. f) Comparison of the test balanced accuracies among different single- and multi-feature classifiers for the HPTC-A dataset. The accuracy values were estimated using 10×10-fold cross validations (error bars=standard errors of the means). g) Multi-dimensional scaling plot showing the phenotypic dissimilarity between all the reference compounds in the HPTC-A dataset based on their Euclidean distances in Fₛ (MDS1/2=the first and second coordinates of the multi-dimensional scaling).

FIG. 6. Shows spatial distribution patterns represented by the best single features. Exemplary immunofluorescence images showing the spatial distribution patterns represented by the best single features (f_{best}) for all three datasets; a) mean entropy of DNA GLCM, b) ratio of the total γH2AX levels at nuclear to the whole cell regions and c) mean correlation of DNA GLCM. Cells with varying (left=low, centre=middle, and right=high) values of the features were shown. The feature values quantified from the images are shown below the respective images.

FIG. 7. Shows average importance values of the final selected features. Average feature importance values estimated using a 10×10 cross validation procedure for the three datasets. Only the top ten features are shown (black bars=final selected features (Ffinal), white bars=other top ten features.) The feature names are shown in the cellXpress format, and a detailed description of the final selected features is included under Table 3.

FIG. 8. Shows a PTC toxicant induced DNA damage response under in vitro conditions. Exemplary immunofluorescence images from the HPTC-B dataset showing the a) DNA and b) γH2AX staining levels of primary human PTCs treated with cyclosporine A (scale bar=20 μm). The quantified values for the CV of DNA, ASM of DNA GLCM, and mean nuclear γH2AX level are shown in the parentheses below the cells. c) Scatter plots showing the raw CV of DNA and mean nuclear γH2AX level of primary PTCs treated with different dosages of cyclosporine A or DMSO (dots=single-cell measurements quantified from the images). Scatter plots showing the maximum responses (Δₘₐₓ) in the CV of DNA and mean nuclear γH2AX level for the d) HPTC-B or e) HK-2 datasets (circles=compounds, Δμ=difference between the mean values of PTC-toxic and non-PTC-toxic compounds, dashed lines=optimum linear-regression fits of the data, r=Pearson's correlation coefficient; all p-values shown were obtained from one-sided t-tests). The six compounds selected for studying the relationships between the DNA damage response and cell death are highlighted. The f) distribution of markers, g) test balanced accuracy, h) test sensitivity, and i) test specificity of the best single and multiple features for all three datasets.

FIG. 9. Shows γH2AX and DNA staining patterns in human HK-2 cells treated with PTC-toxic and non-PTC-toxic compounds. Immunofluorescence microscopy images showing the γH2AX and DNA staining patterns in human HK-2 cells treated with five PTC-toxic compounds (left subpanel), three non-PTC-toxic compounds (right subpanel), and two solvent controls (right subpanel). All images from the same markers have the same exposure times and display intensity ranges (scale bar=20 μm).

FIG. 10. Shows PTC toxicants induce variable cell-death responses. a) Immunofluorescence images showing the γH2AX, ethidium homodimer-III, annexin-V, and cleaved caspase-3 staining levels of primary human PTCs treated with DMSO, cisplatin, and ochratoxin A (scale bar=20 μm). b) Probability density function plots showing how the thresholds (vertical dashed lines) for ethidium-III-, annexin-V-, and caspase-3-positive cells were determined. c) Scatter plots showing the changes in the percentages of ethidium-III-, annexin-V-, or caspase-3-positive cells versus the changes in the mean nuclear γH2AX level (circles=compounds, error bars=standard errors of the means, Δμ=difference between the mean values of PTC-toxic and non-PTC-toxic compounds, dashed lines=optimum linear-regression fits of the data, r=Pearson's correlation coefficient; all p-values shown were obtained from one-sided t-tests).

FIG. 11. A list of reference pulmonotoxic and non-pulmonotoxic compounds and their applications or sources.

FIG. 12. Shows pulmonotoxic soluble and particulate compounds induce changes in the phenotypes of our in vitro pulmonotoxicity model. a) Immunofluorescence images showing human lung cells stained with four fluorescence markers and treated with DMSO (solvent control, top), 2 mM Nitrofurantoin (middle), and 1 mg/mL fumed silica (bottom). These compounds induce changes in the phosphorylation of a DNA damage response marker, γH2AX and the remodeling of actin. Both nitrofurantoin and fumed silica are known to cause pulmonary diseases and silicosis in humans, respectively. b) The phenotypic features were automatically measured from seven subcellular regions.

FIG. 13. Shows human in vivo pulmonotoxicity prediction based on in vitro DNA, γH2AX and actin features of BECs and AVCs. The five best single features f_{best} for soluble and particulate compounds in a) A549 and b) BEAS-2B respectively.

FIG. 14. Immunofluorescence images showing the spatial distribution patterns represented by the f_{best} for soluble compounds in BEAS-2B cells. Images of cells treated with different concentrations of nitrofurantoin (left=control, middle=low, right=high) are shown. The corresponding feature values measured from the cells are shown below the images.

FIG. 15. Immunofluorescence images showing the spatial distribution patterns represented by the f_{best} for particulate compounds in BEAS-2B cells. Images of cells treated with different concentrations of silica particles (left=control, middle=low, right=high) are shown. The corresponding feature values measured from the cells are shown below the images.

### DETAILED DESCRIPTION OF THE INVENTION

In the present invention we used spatial-dependent features to automatically predict in vivo PTC toxicity of xenobiotic compounds. In the examples presented herein we used spatial-dependent features to automatically predict in vivo nephrotoxicity and pulmonotoxicity of xenobiotic compounds.

Bibliographic references mentioned in the present specification are for convenience listed in the form of a list of references and added at the end of the examples. The whole content of such bibliographic references is herein incorporated by reference.

### Definitions

For convenience, certain terms employed in the specification, examples and appended claims are collected here.

As used herein “test sensitivity” as used herein refers to the number of compounds known to be nephrotoxic, pulmonotoxic or toxic to another specific tissue in vivo that are positive according to the test as a percentage of all known nephrotoxic, pulmonotoxic or said another specific tissue toxic compounds tested.

As used herein “test specificity” as used herein refers to the number of compounds known not to be nephrotoxic, pulmonotoxic or toxic to another specific tissue in vivo that are negative according to the test as a percentage of all known non-nephrotoxic, non-pulmonotoxic or said another non-specific tissue toxic compounds tested. For example, said another tissue may comprise cardiac cells, neuronal cells or cancer cells.

As used herein “spatial-dependent phenotypic features” are quantitative, spatial-dependent measurements or statistics of the intensity values of the pixels in the whole- or sub-cellular regions identified from a microscopy image of cells labeled with a biomolecule marker. In other words, the spatial-dependent phenotypic feature characterizes a spatial distribution of biomolecules associated with the cells. The values of such features are dependent on the subcellular localization and spatial distribution of the pixels. The values of such features will change if the locations of the pixels are modified, for example by random shuffling. Otherwise, they are called “spatial-independent phenotypic features”. Examples of spatial-dependent features are textural features, spatial correlations between markers, and intensity ratios of a marker at different subcellular regions. Examples of spatial-independent features are cell count, morphology, and total, mean, standard-deviation, or coefficient-of-variation of the intensities at a whole- or sub-cellular region.

The term “comprising” as used in the context of the invention refers to where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the phenotypic features of the present invention “substantially” comprises the indicated features as “essential” element.

Although the embodiments disclosed herein are directed to predicting whether compounds are nephrotoxic or pulmonotoxic, other types of cell toxicity can also be determined using the processes disclosed herein. For example, cancer cells can be used to screen anti-cancer agents, cardiac cells can be used to investigate cardiotoxicity, dermal cells can be used to investigate dermal toxicity and neuronal cells can be used to investigate neurotoxicity.

In a first aspect of the invention there is provided an in vitro method of predicting whether a test compound will be toxic for a specific cell type in vivo, the method comprising:
(a) contacting at least one test population of cells with the test compound at a single concentration or over a range of concentrations,
(b) labeling and imaging the cells with one or more biomolecular markers,
(c) segmenting the cells and identifying whole-cell regions and one or more subcellular regions from the cells,
(d) determining if cell loss or death has occurred at the highest test concentrations (if so, stop and predict the compound is toxic),
(e) obtaining one or more quantified spatial-dependent and -independent phenotypic features in the test populations,
(f) obtaining multiple dose response curves (DRCs) from the features,
(g) obtaining quantified parameters of the DRCs, and
(h) comparing the quantitated DRC parameters to a reference set of quantitated DRC parameter data; said reference quantitated DRC parameter data being derived from two groups;
   (i) compounds with known in vivo toxicity to the cell type, and (ii) compounds not known to be toxic to the cell type in vivo.

In a preferred embodiment of the invention, the method comprises:
(a) contacting multiple test populations of cells;
(b) labeling and imaging the cells with one or more biomolecular markers,
(c) segmenting the cells and identifying whole-cell regions and one or more subcellular regions from the cells,
(d) determining if cell loss or death has occurred at the highest test concentrations (if so, stop and predict the compound is toxic),
(e) obtaining one or more quantified spatial-dependent and -independent phenotypic features in the test populations,
(f) obtaining multiple dose response curves (DRCs) from the features,
(g) obtaining quantified parameters of the DRCs, and
(h) comparing the quantitated DRC parameters to a reference set of quantitated DRC parameter data; said reference quantitated DRC parameter data being derived from two groups;
   (i) compounds with known in vivo toxicity to the cell type, and (ii) compounds not known to be toxic to the cell type in vivo.

In a preferred embodiment of the method of the invention, said specific cell type is selected from the group comprising renal proximal tubular cells (PTCs), bronchial epithelial cells (BECs), and/or alveolar cells (AVCs).

In a preferred embodiment of the method of the invention, step (h) comprises comparing the quantitated DRC parameters to a reference set of quantitated DRC parameter data; said reference quantitated DRC parameter data being derived from two groups;
in the case of PTCs; (i) compounds with known in vivo PTC toxicity, and (ii) compounds nephrotoxic but not known to be PTC toxic in vivo and compounds not known to be nephrotoxic in vivo; or
in the case of BECs or AVCs; (i) compounds with known in vivo BEC or AVC toxicity, and (ii) compounds pulmonotoxic but not known to be BEC or AVC toxic in vivo and compounds not known to be pulmonotoxic in vivo.

In a preferred embodiment of the method of the invention, said one or more quantitated phenotypic features are associated with characteristics selected from the group comprising DNA damage response, actin filament integrity, whole-cell morphology, and cell count.

In another preferred embodiment of the method of the invention, said one or more phenotypic features are quantitated based on (i) one or more of the spatial-dependent features selected from the group comprising textural features, spatial correlation features, and ratios of markers at different subcellular regions; and (ii) one or more of the spatial-dependent features selected from the group comprising intensity features, cell count, morphology. Textural features may include but are not limited to Haralick's features, Gabor features or Wavelet features

In another preferred embodiment of the method of the invention, the DRC parameters are quantitated using the maximum response value Δₘₐₓ for each feature from a DRC of the test compound. Preferably the median Δₘₐₓ values across three replicate tests are used for prediction analysis.

In another preferred embodiment of the method of the invention, said textural features include one or more of the statistics of the Haralick's grey-level co-occurrence matrix (GLCM) at specific sub- or whole-cellular regions, namely mean correlation of DNA GLCM at the nuclear region; mean entropy of DNA GLCM at the nuclear region; mean angular second moment of DNA GLCM at the nuclear region; standard deviation of the sum variance of DNA GLCM at the nuclear region; mean sum entropy of actin GLCM at the whole cell region; mean entropy of actin GLCM at the whole cell region; standard deviation of the information measure of correlation 2 of the DNA damage response marker histone H2AX phosphorylated on Serine 139 (γH2AX) γH2AX GLCM at the whole-cell region; and mean sum average of γH2AX GLCM at the whole cell region. Preferably, the actin marker is F-actin.

In another preferred embodiment of the method of the invention, said staining intensity feature is selected from one or more of the group comprising normalized spatial correlation coefficient between DNA and actin intensities at the whole cell region; total actin intensity level at the inner cytoplasmic region; normalized spatial correlation coefficient between DNA and γH2AX intensities at the whole cell region; and coefficient of variation of the DNA intensity at the nuclear region.

In another preferred embodiment of the method of the invention, said staining intensity ratio feature is selected from one or more of the group comprising ratio of the total γH2AX to DNA intensities at the whole cell region; the ratio of the total γH2AX to actin intensities at the nuclear region; and ratio of the total γH2AX intensity levels at the nuclear region to the whole cell region.

In another preferred embodiment of the method of the invention, the said one or more phenotypic features are selected from the group comprising mean sum entropy of the actin GLCM at the whole-cell region; coefficient of variation (CV) of the DNA intensity at the nuclear region; mean entropy of the actin GLCM at the whole-cell region; and mean angular second moment (ASM) of DNA GLCM at the nuclear region.

In another preferred embodiment of the method of the invention, the said one or more phenotypic features are selected from the group comprising total actin intensity level at the inner cytoplasmic region; mean angular second moment (ASM) of DNA GLCM at the nuclear region; standard deviation of the information measure of correlation 2 of γH2AX GLCM at the whole-cell region; and cell count.

In another preferred embodiment of the method of the invention, the said one or more phenotypic features are selected from the group comprising normalized spatial correlation coefficient between DNA and γH2AX intensities at the whole-cell region; normalized spatial correlation coefficient between DNA and actin intensities at the whole-cell region; mean sum average of γH2AX GLCM at the whole-cell region; ratio of the total γH2AX to DNA intensities at the whole-cell region; and standard deviation of the sum variance of DNA GLCM at the nuclear region.

In another preferred embodiment of the method of the invention, the said one or more phenotypic features are selected from the group comprising mean entropy of the DNA GLCM at the nuclear region; ratio of the total γH2AX intensity levels at the nuclear region to the whole-cell region; mean correlation of actin GLCM; and mean correlation of DNA GLCM at the nuclear region.

In another preferred embodiment of the method of the invention, the said one or more phenotypic features consist of the group mean sum entropy of the actin GLCM at the whole-cell region; coefficient of variation (CV) of the DNA intensity at the nuclear region; mean entropy of the actin GLCM at the whole-cell region; and mean angular second moment (ASM) of DNA GLCM at the nuclear region.

In another preferred embodiment of the method of the invention, the said one or more phenotypic features consist of the group total actin intensity level at the inner cytoplasmic region; mean angular second moment (ASM) of DNA GLCM at the nuclear region; standard deviation of the information measure of correlation 2 of γH2AX GLCM at the whole-cell region; and cell count.

In another preferred embodiment of the method of the invention, the said one or more phenotypic features consist of the group normalized spatial correlation coefficient between DNA and γH2AX intensities at the whole-cell region; normalized spatial correlation coefficient between DNA and actin intensities at the whole-cell region; mean sum average of γH2AX GLCM at the whole-cell region; ratio of the total γH2AX to DNA intensities at the whole-cell region; and standard deviation of the sum variance of DNA GLCM at the nuclear region.

In another preferred embodiment of the method of the invention, step (b) comprises obtaining the quantitated phenotypic features using fluorescent, isotope, or colorimetric markers and imaging techniques. The markers may be used to detect DNA, chromatin, actin filaments, and generally stain the whole cell. For example, these biomolecules can be genetically labeled by tagging them with fluorescent proteins. For example, an antibody can be used to detect the DNA damage response marker histone H2AX phosphorylated on Serine 139 (γH2AX), or the cells can be stained with 4′,6-diamidino-2-phenylindole (DAPI) or 2,5′-Bi-1H-benzimidazole (Hoechst 33342) to label the DNA; rhodamine phalloidin or Dylight™ 554 phalloidin to label the actin cytoskeleton; and HCS CellMask™ deep red stain or whole cell stain red; a reactive dye that binds to cell surfaces and contents to provide complete and even visualization of fixed cells in fluorescence imaging. The whole cell stain may be used to identify and count individual cells and to define the cell region in which image analysis is applied.

Preferably, said imaging techniques comprise high-throughput microscopy image capture which may be followed by computer-assisted quantitation and processing. A representative example is disclosed herein.

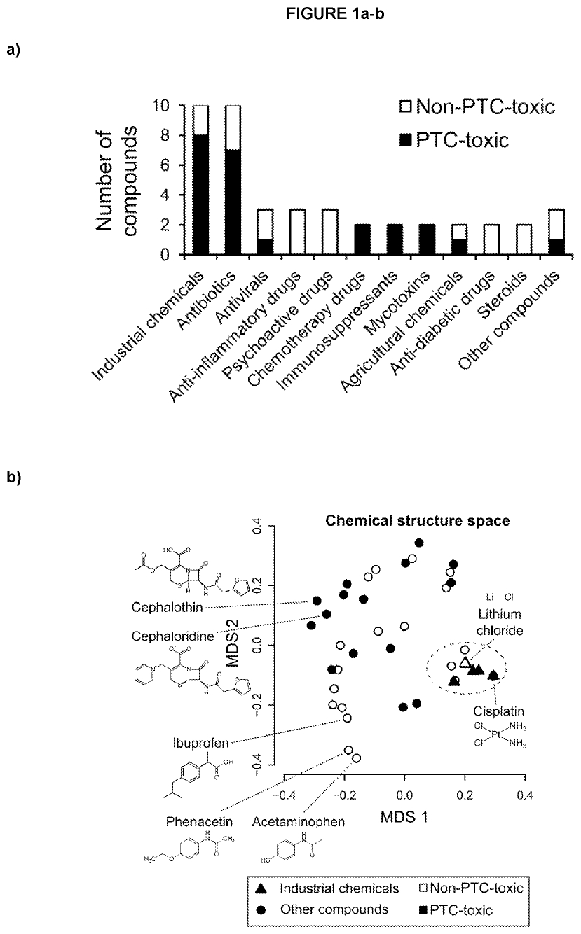

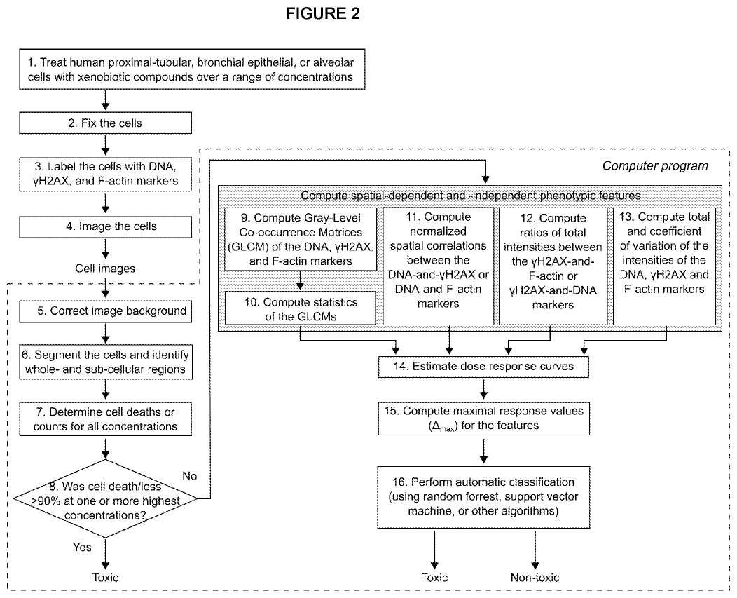

In another preferred embodiment of the method of the invention, cell toxicity, more preferably nephrotoxicity or pulmonotoxicity is predicted using any suitable supervised learning algorithm. Preferably, the prediction is performed using a random-forest algorithm (see Examples and FIGS. 1 and 2), a support vector machine, or a neural network. More preferably, the prediction is performed using a random-forest algorithm.

In another preferred embodiment of the method of the invention, the at least one test population of cells and, more preferably, the renal proximal tubular cells, BECs and AVCs, may be derived from somatic cells. More preferably, the at least one test population of cells comprising renal proximal tubular cells, BECs and AVCs are derived from mammalian somatic cells and are primary cells or cells from a stable cell line.

In another preferred embodiment of the method of the invention, the renal proximal tubular cells are human primary renal proximal tubular cells, HK-2 cells or any other suitable cell line known in the art.

In another preferred embodiment of the method of the invention, the at least one test population of cells are human primary cells, immortalized cells, embryonic-stem-cell-derived cells, induced-pluripotent-stem-cell-derived cells, or any other suitable cell line known in the art.

In another preferred embodiment of the method of the invention, the BECs and AVCs are human primary alveolar or bronchial epithelial cells, immortalized cells, embryonic-stem-cell-derived cells, induced-pluripotent-stem-cell-derived cells, or any other suitable cell line known in the art.

In another preferred embodiment of the method of the invention, said contacting is performed over a period of time of at least 1-48 hours or more. Preferably, the cells are contacted with test compound for a period of about 8-24 hours, more preferably a period of about 16 hours.

The actual concentration of test compound to contact the cells with will depend on the nature of the specific compound to be tested. In a preferred embodiment of the method of the invention, said contacting comprises adding the test compound to the test population of renal proximal tubular cells at a concentration of about 1 μg/ml to about 1000 μg/ml; or to the test population of bronchial epithelial cells and alveolar cells at about 31 μM to about 2 mM for soluble compounds and about 16μg/mL to about 1 mg/mL for particulate compounds. Preferably, a concentration is used to achieve a maximum response value Δₘₐₓ for each feature from a dose response curve of the test compound. It is possible to use a single dose of test compound in the method according to the invention; although it is preferable to test a compound over a range of concentrations simultaneously.

In another embodiment, there is provided a computer-implemented method of predicting in vivo cell toxicity of a test compound using a test population of the cells subjected to the test compound in vitro, the method comprising:

(a) receiving, by a computer processor, an image of the test population of the cells;

(b) extracting, by the computer processor, one or more spatial-dependent phenotypic features associated with the test population of cells from the image, the one or more spatial-dependent phenotypic feature characterizing a spatial distribution of biomolecules associated with the cells;

(c) obtaining one or more quantitated DRC parameters describing the DRCs of the respective one or more spatial-dependent phenotypic features; and

(d) inputting said one or more quantitated DRC parameters to a predictive model to generate a prediction of in vivo cell toxicity of the test compound.

In a preferred embodiment of the method of the invention, the cells are renal proximal tubular cells (PTCs), bronchial epithelial cells (BECs), or alveolar cells (AVCs).

In a preferred embodiment of the method of the invention, the one or more spatial-dependent phenotypic features are selected from the group comprising features characterizing DNA structure alterations, chromatin structure alterations and Actin filament structure alterations of the cells.

In another example, the method may further comprise extracting one or more spatial-independent phenotypic features associated with the test population of cells, wherein obtaining the one or more quantitated DRC parameters further using the one or more spatial-independent phenotypic features.

In yet another example as shown in FIG. 2, the level of cell death due to the toxicity of the test compound is assessed before computing the quantitated phenotypic features. In particular, the method may comprise a step of assessing whether a level of cell death at one or more of the highest concentrations of the test compound has met a pre-determined criterion, such as whether the level of cell death exceeds a pre-determined threshold, before performing steps (c) and (d). For example, this method may be applied to cell data relating to a specific type of cells, such as lung cells.

In a preferred embodiment of the method of the invention, step (d) comprises classifying the test compound to either toxic or non-toxic for the cells.

Another aspect of the invention provides a computer system having a computer processor and a data storage device, the data storage device storing non-transitory instructions operative by the processor to perform a computer-implemented method according to any aspect of the invention.

Another aspect of the invention provides a non-transitory computer-readable medium, the computer-readable medium having stored thereon program instructions for causing at least one processor to perform a computer-implemented method according to any aspect of the invention.

A person skilled in the art will appreciate that the present invention may be practiced without undue experimentation according to the method given herein. The methods, techniques and chemicals are as described in the references given or from protocols in standard biotechnology, cell biology and immunohistochemistry text books.

### EXAMPLES

### Reference Compounds for Nephrotoxicity Study

For the HPTC-A dataset (DNA/RelA/actin/WCS), we used 44 xenobiotic compounds. The “PTC-toxic” group had 24 nephrotoxicants known to damage human proximal tubular cells (PTCs), and the “non-PTC-toxic” group had 12 nephrotoxicants not known to damage PTCs and 8 non-nephrotoxicants (detailed information on the PTC toxicity of most of the compounds can be found in our reports (Li et al., Mol Pharm 11: 1982-1990 (2014); Kandasamy et al., Sci Rep. doi: 10.1038/srep12337 (2015)). For the HPTC-B and HK-2 datasets (DNA/γH2AX/actin/WCS), 42 of the compounds were used (excluding lead acetate and hydrocortisone). The compounds were dissolved in either DMSO at a stock concentration of 50 mg/mL, or water at a stock concentration of 10 mg/mL. The full list of reference compounds and their sources, solvents, and known human kidney and liver toxicity are provided in Table 1.

TABLE 1
Reference nephrotoxic compounds.

| | CAS | PTC- | Nephro | Hepato | | HPTC- | HPTC- | |
|---|---|---|---|---|---|---|---|---|
| Drug name | number | toxic | toxic | toxic | Category | A | B/HK2 | Solvent |
| 5-Fluoro- | 51-21-8 | 1 | 1 | 1 | Chemo- | Y | Y | DMSO |
| uracil | | | | | therapy | | | |
| | | | | | drugs | | | |
| Acarbose | 56180- | 0 | 0 | 1 | Anti-diabetic | Y | Y | water |
| | 94-0 | | | | drugs | | | |
| Acetamino- | 103-90- | 0 | 1 | 1 | Anti- | Y | Y | water |
| phen | 2 | | | | inflammatory | | | |
| | | | | | drugs | | | |
| Aristolochic | 313-67- | 1 | 1 | 1 | Herbs | Y | N | DMSO |
| acid | 7 | | | | | | | |
| Arsenic(III) | 1327- | 1 | 1 | 1 | Industrial | Y | Y | water |
| oxide | 53-3 | | | | chemicals | | | |
| Bismuth(III) | 1304- | 1 | 1 | 1 | Industrial | Y | Y | water |
| oxide | 76-3 | | | | chemicals | | | |
| Cadmium(II) | 10108- | 1 | 1 | 1 | Industrial | Y | Y | water |
| chloride | 64-2 | | | | chemicals | | | |
| Cephaloridine | 50-59-9 | 1 | 1 | 0 | Antibiotics | Y | Y | water |
| Cephalosporin | 61-24-5 | 1 | 1 | 0 | Antibiotics | Y | Y | water |
| C | | | | | | | | |
| Cephalothin | 153-61- | 1 | 1 | 0 | Antibiotics | Y | Y | water |
| | 7 | | | | | | | |
| Ciprofloxacin | 85721- | 0 | 1 | 1 | Antibiotics | Y | Y | water |
| | 33-1 | | | | | | | |
| Cisplatin | 15663- | 1 | 1 | 0 | Chemo- | Y | Y | DMSO |
| | 27-1 | | | | therapy drugs | | | |
| Citrinin | 518-75- | 1 | 1 | 0 | Mycotoxins | Y | Y | DMSO |
| | 2 | | | | | | | |
| Copper(II) | 7447- | 1 | 1 | 1 | Industrial | Y | Y | water |
| chloride | 39-4 | | | | chemicals | | | |
| Cyclosporin | 59865- | 1 | 1 | 1 | Immuno- | Y | Y | DMSO |
| A | 13-3 | | | | suppressants | | | |
| Dexa- | 50-02-2 | 0 | 0 | 0 | Steroids | Y | Y | water |
| methasone | | | | | | | | |
| Ethylene | 107-21- | 0 | 1 | 0 | Industrial | Y | Y | water |
| glycol | 1 | | | | chemicals | | | |
| Furosemide | 54-31-9 | 0 | 1 | 1 | Other drugs | Y | Y | DMSO |
| Gentamicin | 1403- | 1 | 1 | 0 | Antibiotics | Y | Y | water |
| | 66-3 | | | | | | | |
| Germanium | 1310- | 1 | 1 | 0 | Industrial | Y | Y | water |
| (IV) oxide | 53-8 | | | | chemicals | | | |
| Glycine | 56-40-6 | 0 | 0 | 0 | Food | Y | Y | water |
| | | | | | additives | | | |
| Gold(I) | 10294- | 1 | 1 | 1 | Industrial | Y | Y | water |
| chloride | 29-8 | | | | chemicals | | | |
| Hydro- | 50-23-7 | 0 | 0 | 0 | Steroids | Y | Y | DMSO |
| cortisone | | | | | | | | |
| Ibuprofen | 15687- | 0 | 1 | 1 | Anti- | Y | Y | DMSO |
| | 27-1 | | | | inflammatory | | | |
| | | | | | drugs | | | |
| Lead(IV) | 546-67- | 1 | 1 | 1 | Industrial | Y | Y | DMSO |
| acetate | 8 | | | | chemicals | | | |
| Levodopa | 59-92-7 | 0 | 0 | 0 | Psycho- | Y | Y | water |
| | | | | | active drugs | | | |
| Lincomycin | 154-21- | 0 | 1 | 0 | Antibiotics | Y | Y | water |
| | 2 | | | | | | | |
| Lindane | 58-89-9 | 0 | 1 | 0 | Agricultural | Y | Y | DMSO |
| | | | | | chemicals | | | |
| Lithium | 7447- | 0 | 1 | 0 | Industrial | Y | Y | water |
| chloride | 41-8 | | | | chemicals | | | |
| Melatonin | 73-31-4 | 0 | 0 | 0 | Psycho- | Y | Y | DMSO |
| | | | | | active drugs | | | |
| Metformin | 657-24- | 0 | 1 | 0 | Anti-diabetic | Y | Y | water |
| hydrochloride | 9 | | | | drugs | | | |
| Ochratoxin A | 303-47- | 1 | 1 | 0 | Mycotoxins | Y | Y | DMSO |
| | 9 | | | | | | | |
| Paraquat | 1910- | 1 | 1 | 1 | Agricultural | Y | Y | water |
| | 42-5 | | | | chemicals | | | |
| Phenacetin | 62-44-2 | 0 | 1 | 1 | Anti- | Y | Y | DMSO |
| drugs | | | | | inflammatory | | | |
| Potassium | 7778- | 1 | 1 | 1 | Industrial | Y | Y | water |
| dichromate | 50-9 | | | | chemicals | | | |
| Puromycin | 53-79-2 | 1 | 1 | 1 | Antibiotics | Y | Y | water |
| Ribavirin | 36791- | 0 | 0 | 0 | Antivirals | Y | Y | water |
| | 04-5 | | | | | | | |
| Rifampicin | 13292- | 1 | 1 | 1 | Antibiotics | Y | Y | DMSO |
| | 46-1 | | | | | | | |
| Tacrolimus | 104987- | 1 | 1 | 0 | Immuno- | Y | Y | DMSO |
| | 11-3 | | | | suppressants | | | |
| Tenofovir | 147127- | 1 | 1 | 0 | Antivirals | Y | N | water |
| | 20-6 | | | | | | | |
| Tetracycline | 60-54-8 | 1 | 1 | 1 | Antibiotics | Y | Y | water |
| Triiodo- | 6893- | 0 | 0 | 0 | Psychoactive | Y | Y | DMSO |
| thyronine | 02-3 | | | | drugs | | | |
| Valacyclovir | 124832- | 0 | 1 | 1 | Antivirals | Y | Y | water |
| | 26-4 | | | | | | | |
| Vancomycin | 1404- | 0 | 1 | 1 | Antibiotics | Y | Y | water |
| | 90-6 | | | | | | | |

### Cell Culture and Compound Treatment

For both the HPTC-A and -B datasets, we used three different batches of primary human PTCs from three different donors. Two of them (HPTC1 and HPTC10; Lot #58488852 and #61247356, respectively) were bought from the American Type Culture Collection (ATCC, Manassas, Va., USA). The third batch of cells (HPTC6) was isolated from a human nephrectomy sample (National University Health System, Singapore). Only normal tissues without aberrant pathological changes, as determined by a pathologist, were used. Ethics approvals for the work with primary human kidney samples (DSRB-E/11/143) and cells (NUS-IRB Ref. Code: 09-148E) were obtained. All three batches of primary PTCs were cultured in renal epithelial cell basal medium (ATCC) supplemented with renal epithelial cell growth kit (ATCC) and 1% penicillin/streptomycin (Gibco, Carlsbad, Calif., USA). Only passages (P) 4 and P5 of primary PTCs were used in this study. For the HK-2 dataset, the HK-2 cell line (ATCC) was maintained in Dulbecco's modified eagle medium (DMEM) supplemented with 10% foetal bovine serum (FBS) (Gibco) and 1% penicillin/streptomycin.

Cells were seeded into 384-well black plates with transparent bottom (Greiner, Kremsmünster, Austria). All cells were cultured for 3 days to achieve the formation of a differentiated renal epithelium before overnight drug treatment (16 hours) (Li et al., Toxicol Res 2: 352-365 (2013)). The dosages of the tested compounds were 1.6, 16, 63, 125, 250, 500, 1000 μg/mL. Positive, negative, and vehicle controls (DMSO or water, depending on the solvent of the tested compounds) and untreated cells were included on each plate. Four technical replicates were performed for each compound and dosage.

### Immunostaining

After compound treatment for 16 hours, cells were fixed using 3.7% formaldehyde in phosphate-buffered saline (PBS). The cells were blocked for 1 hour with PBS containing 5% bovine serum albumin (BSA) and 0.2% Triton X-100. The samples were incubated with a mouse monoclonal antibody to γH2AX (phospho S139) (Abcam, Cambridge, Mass., USA) at 2 μg/mL, or a rabbit polyclonal antibody to RelA (Abcam) at 1 μg/mL for 1 hour at room temperature. Subsequently, the cells were incubated with a goat anti-mouse secondary antibody conjugated to Alexa488 (Abcam) or a goat anti-rabbit secondary antibody conjugated to Alexa488 (Life Technologies, Carlsbad, Calif., USA) at 5 μg/mL. Finally, the cells were stained with DAPI (Merck Millipore, Darmstadt, Germany) at 4 ng/mL, rhodamine phalloidin (Life Technologies) and whole cell stain red (Life Technologies).

### Apoptosis and Necrosis Assays

Cells were seeded into 96-well black plates with transparent bottom (Falcon, Corning, N.Y., USA) and cultured for 3 days before overnight drug treatment (16 hours). They were treated with cisplatin, cyclosporin A, ochratoxin A, lincomycin, lithium chloride and ribavirin at 1000 μg/mL. Untreated cells and vehicle controls (DMSO and water) were included on each plate as well as positive (25 μg/mL arsenic(III) oxide) and negative (100 μg/mL dexamethasone) controls. Three technical replicates were performed for each treatment condition.

Cleaved caspase-3 (Abcam) and apoptotic/necrotic/healthy cells detection kits (PromoKine, Heidelberg, Germany) were used to identify apoptotic and necrotic cells. For cleaved caspase-3, the same immunostaining protocol as outlined above was used. The rabbit polyclonal anti-cleaved-caspase-3 antibody was diluted in blocking buffer and incubated with fixed cells for 1 hour in room temperature. The cells were then incubated with a goat anti-rabbit secondary antibody conjugated to Alexa 488 at 5 μg/mL. Finally, the cells were counterstained with DAPI at 4 μg/mL and whole cell stain red. For the apoptotic/necrotic/healthy cells detection kit, the protocols provided by manufacturer were used.

### Image Acquisition

Imaging was performed with a 20× objective using the ImageXpress Micro XLS system (Molecular Devices, Sunnyvale, Calif., USA). Four different channels were used to image DAPI, Alexa 488, Texas Red, and Cy5 fluorescence. Nine sites per well were imaged. The images were saved in 16-bit TIFF format.

### Image Segmentation and Feature Extraction

To reduce non-uniform background illuminations, we corrected the images using the “rolling ball” algorithm implemented in ImageJ (NIH, v1.48) (Sternberg Computer 16: 22-34 (1983)). Cell segmentations and feature measurements were performed using the cellXpress software platform (Bioinformatics Institute, v1.2) (Laksameethanasan et al., BMC Bioinformatics 14 Suppl 16: S4 (2013)). We extracted 129 features, which include 78 Haralick's texture features, 29 intensity features, 9 intensity-ratio features, 6 correlation features, 6 morphology features and cell count from the images.

### Haralick's Texture Features

The mathematical definitions of all Haralick's texture features were described in Haralick's original paper (Haralick et al., IEEE Trans Syst Man Cybern SMC-3: 610-621 (1973)). Here, we only provide mathematical definitions for the Haralick's features included in our final feature sets. A grey-level co-occurrence matrix (GLCM) is a matrix that describes the distribution of co-occurring grey-level values at a given offset (Δx, Δy) in an Nₓ×N_{y} image, I(x, y), with N_{g} grey levels. In our notations, x and y are the row and column indices, respectively. The GLCM matrix is defined by

$${GLCM}_{Δ x , Δ y} ( i , j ) = {\sum }_{x = 1}^{{N}_{x}} {\sum }_{y = 1}^{{N}_{y}} \{ \begin{matrix}1 , & if I ( x , y ) = i and I ( x + Δ x , y + Δ y ) = j \\ 0 , & \text{otherwise}\end{matrix} ,$$

where i and j are the grey-level or intensity values of the image. The normalized GLCM matrix is

$$p ( i , j , Δ x , Δ y ) = \frac{{GLCM}_{Δ x , Δ y} ( i , j )}{{\sum }_{i = 1}^{{N}_{g}} {\sum }_{j = 1}^{{N}_{g}} {GLCM}_{Δ x , Δ y} ( i , j )} .$$

Then, we have the marginal and sum probability matrices to be

$${p}_{x} ( j , Δ x , Δ y ) = {\sum }_{i = 1}^{{N}_{g}} p ( i , j , Δ x , Δ y ) , \\ {p}_{y} ( i , Δ x , Δ y ) = {\sum }_{j = 1}^{{N}_{g}} p ( i , j , Δ x , Δ y ) , and$$

$${p}_{x + y} ( k , Δ x , Δ y ) = {{\sum }_{i = 1}^{{N}_{g}} {\sum }_{j = 1}^{{N}_{g}}}_{i + j = k} p ( i , j , Δ x , Δ y ) , where k = 2 , 3 , ⋯ , 2 , {N}_{g} .$$

The Haralick's features are
a) Angular second moment:

$${f}_{ASM} ( Δ x , Δ y ) = {\sum }_{i} {\sum }_{j} {\{ p ( i , j , Δ x , Δ y ) \}}^{2}$$

b) Correlation:

$${f}_{COR} ( Δ x , Δ y ) = \frac{1}{{σ}_{x} {σ}_{y}} {\sum }_{i} {\sum }_{j} ( ij ) p ( i , j , Δ x , Δ y ) - {μ}_{x} {μ}_{y} ,$$

where μₓ, μ_{y}, σₓ and σ_{y} are the means and standard deviations of pₓ(j, Δx, Δy) and p_{y}(i, Δx, Δy), respectively.
c) Sum average:

$${f}_{SA} ( Δ x , Δ y ) = {\sum }_{k = 2}^{2 {N}_{g}} k {p}_{x + y} ( k , Δ x , Δ y )$$

d) Sum variance:

$${f}_{SV} ( Δ x , Δ y ) = {\sum }_{k = 2}^{2 {N}_{g}} {( k - {f}_{SA} ( Δ x , Δ y ) )}^{2} {p}_{x + y} ( k , Δ x , Δ y )$$

e) Sum entropy:

$${f}_{SE} ( Δ x , Δ y ) = - {\sum }_{k = 2}^{{N}_{g}} {p}_{x + y} ( k , Δ x , Δ y ) log [ {p}_{x + y} ( k , Δ x , Δ y ) ]$$

f) Entropy:

$${f}_{E} ( Δ x , Δ y ) = - {\sum }_{i} {\sum }_{j} p ( i , j , Δ x , Δ y ) log [ p ( i , j , Δ x , Δ y ) ]$$

g) Information measure of correlation 2:

$${f}_{IMC 2} ( Δ x , Δ y ) = \sqrt{\vert 1 - exp [ - 2 ( HXY 2 - {f}_{E} ( Δ x , Δ y ) ) ] \vert} , where$$

$$HXY 2 = - {\sum }_{i} {\sum }_{j} {P}_{x} ( j , Δ x , Δ y ) {p}_{y} ( i , Δ x , Δ y ) log [ {p}_{x} ( j , Δ x , Δ y ) {p}_{y} ( i , Δ x , Δ y ) ]$$

In our study, the images were the bounding boxes around the segmented cells with all the background pixels set to zero. We quantized the images into N_{g}=256 grey levels, and computed all the Haralick's features for 0 degree (Δx=0, Δy=1), 45 degree (Δx=1, Δy=1), 90 degree (Δx=1, Δy=0), and 135 degree (Δx=1, Δy=−1) offsets. For each feature, the mean and standard deviation of the feature across all the offset values were used. We have implemented the extraction procedures for all the features using C++ in the cellXpress software platform (Bioinformatics Institute, v1.2) (Laksameethanasan et al., BMC Bioinformatics 14 Suppl 16: S4 (2013)).

### Concentration Response Curve and Δₘₐₓ Estimations

After feature extraction, we divided the values of a feature at all the tested compound concentrations by the values of the feature under the corresponding vehicle control conditions. Then, the ratios were log 2-transformed (Δ). All further data analyses, including building concentration response curves and toxicity classifiers, were performed using customized scripts under the R statistical environment (the R foundation, v3.0.2) and the Windows 7 operating system (Microsoft, USA).

For each feature, we estimated its concentration response curve (DRC) using a standard log-logistic model:

$$Δ ( x , ( b , c , d , e ) ) = \frac{d - c}{1 + exp \{ b ( log ( x ) - log ( e ) ) \}} ,$$

where x is the xenobiotics compound concentration, e is the response half-way between the lower limit c and upper limit d, and b is the relative slope around e. We used the “drc” library (v 2.3-96) under the R environment to fit the values of b, c, d, and e. After that, the maximum response values (Δₘₐₓ) were determined using the estimated response curves. In theory, Δₘₐₓ should be equal to the upper limit d. However, in practice, the responses of some compounds may not plateau even at the highest tested dosages, and therefore the estimated d value may not be accurate. Instead, we fixed Δₘₐₓ to be the response value at 5 mM, which was around the highest tested concentrations for most of the our compounds. Finally, the median values of Δₘₐₓ across the three biological replicates were computed. The final result was a 129×44 (or 42) matrix of Δₘₐₓ values, which were used for training and testing the classifiers. Each column of the matrix was a feature vector, fᵢ, where i=1, 2, . . . , 129.

### Feature Normalization

Before data classification, each feature vector fᵢ was normalized to the same range [−1, 1]:

$${f}_{i} ← 2 \frac{( {f}_{i} - {f}_{min} )}{{f}_{max} - {f}_{min}} - 1 ,$$

where fₘᵢₙ and vₘₐₓ are the minimum and maximum values of the feature. To ensure the training and test datasets were independent to each other, these two normalization coefficients were estimated only using the training data, but applied to both training and test datasets.

### Random Forest Classification

We used the random-forest algorithm (Breiman Mach Learn 45: 5-32 (2001)) to predict xenobiotic-induced nephrotoxicity, because we have previously shown that the algorithm outperforms other commonly-used classifiers, including support vector machine, k-nearest neighbors and naïve Bayes (Su et al., BMC Bioinformatics 15: S16 (2014)). A random forest has two main parameters: Nₜᵣₑₑ and Nₜᵣᵢₐₗ. The first parameter specifies the number of decision trees built, and the second parameter specifies the number of random features used at each level of the decision trees. During cross validation, we automatically determine the optimum classifier parameters using a grid search for Nₜᵣₑₑ={10,50,150,250,400,500} and Nₜᵣᵢₐₗ={1,2,3,4,5}. A series of temporary random forests were trained using all the possible combinations of parameters based on a training dataset X̅′_{training}, and the test accuracies of these combinations were estimated based on an independent test dataset X̅′_{FStest}. The combination of Nₜᵣₑₑ and Nₜᵣᵢₐₗ with the highest test accuracy value were selected to train a final classifier, whose performance would then be estimated using a third independent test dataset X′_{RFtest}. We used the “randomForest” library (v4.6-10) under the R environment.

### Automated Feature Selection

We used a greedy search algorithm, namely recursive feature elimination (RFE) (Loo et al., Nat Methods 4: 445-453 (2007)), to select a subset of features from all the extracted features Fₐₗₗ={f₁, f₂, . . . , fₘ_{ₐₗₗ}}.

The main idea is to start with all the features; iteratively rank the current feature set, remove the least important feature subset, evaluate the accuracy accⱼ of the retained feature subset Fⱼ; and finally select the feature subset with the highest accuracy. To reduce data overfitting, the ranking and evaluation of feature subsets were performed in two independent datasets, {X̅′_{training}, X̅′_{FStest}} and X̅′_{RFtest}, respectively. We ranked features based on their importance values estimated by the random forest algorithm by permuting the out-of-bag data and features (Breiman Mach Learn 45: 5-32 (2001)).

In datasets with small sample sizes, the accⱼ curve (as a function of Fⱼ) may not be smooth. Thus, the global maxima of accⱼ may not be a robust criterion for selecting the final feature subset. Instead, we designed an automated procedure to select a feature subset using Gaussian mixture modeling (GMM) (Trevor Hastie et al., Data Mining, Inference, and Prediction, 2nd edn. Springer (2009)). We clustered all the accⱼ values into 2-4 groups. Each of them was modeled as a Gaussian distribution. Then, we selected the smallest feature subset in the group with the highest average prediction accuracy. The optimum number of groups was also automatically determined based on the Bayesian information criterion (BIC), BIC=−2Lₘ+N_{d}log(Nₛ), where Nₛ is the sample size, Lₘ is the maximum log-likelihood computed by the GMM algorithm, and N_{d} is the number of the parameters.

### Classification Performance Estimation

We used a stratified 10-fold cross validation procedure (Trevor Hastie et al., Data Mining, Inference, and Prediction, 2nd edn. Springer (2009)) to estimate the PTC-toxicity prediction performance of our phenotypic features.

The procedure has two main cross-validation loops. The first cross-validation loop aims to identify an optimum feature subset F_{final}, while the second cross-validation loop aims to estimate the generalized prediction performance of F_{final}. To keep the training and test data independent from each other, we divided all the treatment conditions into four non-overlapping sets, X_{training}(Fₐₗₗ), X_{FStest}(Fₐₗₗ), X_{RFtest}(Fₐₗₗ), and Xₜₑₛₜ(Fₐₗₗ). Furthermore, the normalization coefficients and classifier parameters were always estimated based on the training datasets only, but applied to both training and test datasets.

We used the following performance measurements:

$$\text{Sensitivity} = \frac{TP}{TP + FN} \times 100 \% , \\ \text{Specificity} = \frac{TN}{TN + FP} \times 1 \; 00 \% , and$$

$$\text{Balanced accuracy} ( acc ) = \frac{\text{Sensitivity} + \text{Specificity}}{2} ,$$

where TP is the number of true positives, TN is the number of true negatives, FP is the number of false positives and FN is the number of false negatives. The same performance estimation procedure was used for HPTC-A, HPTC-B and HK-2 datasets.

### Multi-Dimensional Scaling Plots

To compare the compounds in the chemical structure space, we used the ChemmieR library to compute the pairwise Tanimoto coefficients between the structures of all the reference compounds. To compare the compounds in the phenotypic feature space, we first scaled all the phenotypic features to the same range [0, 1], and then computed the pairwise Euclidean distances between the feature values of all the reference compounds. Finally, we used the cmdscale function (Torgerson, Psychometrika 17: 401-419 (1952)) in the R environment to generate the multi-dimensional scaling plots.

### Reference Compound List

To make our computational models more comprehensive, we increased the number of reference kidney xenobiotic compounds to 44 (Table 1), among which 38 compounds were previously used in our IL-6/8-based models (Li et al., Toxicol Res 2: 352-365 (2013); Li et al., Mol Pharm 11: 1982-1990 (2014); Su et al., BMC Bioinformatics 15: S16 (2014); Kandasamy et al., Sci Rep. doi: 10.1038/srep12337 (2015)). These reference compounds included commonly used industrial chemicals, antibiotics, antivirals, chemotherapy drugs, mycotoxins, agricultural chemicals and other compounds (FIG. 1a). They were divided into two groups based on their known in vivo toxicity from published clinical and/or animal studies (detailed information for most of the compounds can be found in our previous reports (Li et al., Mol Pharm 11: 1982-1990 (2014); Kandasamy et al., Sci Rep. doi: 10.1038/srep12337 (2015)). The “PTC-toxic” group had 24 nephrotoxicants known to damage PTCs, and the “non-PTC-toxic” group had 12 nephrotoxicants not known to damage PTCs in humans and 8 compounds not known to damage the human kidney. Furthermore, ten and eight compounds in the PTC-toxic and non-PTC-toxic groups, respectively, were known to be hepatotoxic (Table 1). This design of reference compound list ensured that we would not favor phenotypic features for general or non-PTC-specific toxicity. Our binary categorization of the compounds simplified the prediction problem and allowed us to use well-established prediction performance criteria to evaluate different phenotypic features and cell types. Our reference compounds had diverse chemical structures, and we found no obvious structural difference between the PTC-toxic and non-PTC-toxic compounds (FIG. 1b). For example, all of the industrial chemicals were clustered together in the chemical space irrespective of their known PTC toxicity (dashed line in FIG. 1b). Most of them were metallic compounds, such as cisplatin (PTC-toxic) and lithium chloride (non-PTC-toxic), which have very simple and thus hard-to-differentiate molecular structures. We treated primary human PTCs from three different donors with these compounds in seven different doses (1.6-1000 μg/mL) for 16 hours.

### Reference Compounds for Pulmonotoxicity Study

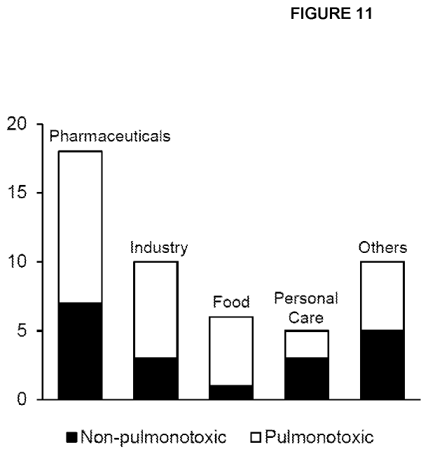

For the pulmonotoxicity study we used 49 xenobiotic compounds, of which 39 were soluble and 10 were particulate compounds (Table 2). The 19 pulmonotoxic and 30 non-pulmonotoxic compounds were selected based on published in vivo and/or clinical data. These reference compounds included commonly found or used pharmaceuticals, industrial chemicals, food, personal care and others such as cigarette smoke etc (FIG. 11).

TABLE 2
Reference pulmonotoxic compounds.

| | CAS | | Pulmono | | |
|---|---|---|---|---|---|
| Xenobiotics | number | Particles | toxic | Category | Solvent |
| Amidoarone | 19774-82-4 | 0 | 1 | Pharma- | DMSO |
| hydrochloride | | | | ceuticals | |
| Asbestos | 12001-29-5 | 1 | 1 | Others | Water |
| (Chrysotile) | | | | | |
| Barium sulfate | 7727-43-7 | 1 | 1 | Industry | Water |
| Bicalutamide | 90357-06-5 | 0 | 0 | Pharma- | DMSO |
| | | | | ceuticals | |
| Bleomycin | 9041-93-4 | 0 | 1 | Pharma- | DMSO |
| sulfate | | | | ceuticals | |
| Butylated | 128-37-0 | 0 | 0 | Food | EtOH |
| hydroxytoluene | | | | | |
| 1,3-Butylene | 107-88-0 | 0 | 0 | Personal | Water |
| glycol | | | | care | |
| Cadmium (II) | 9041-93-4 | 0 | 1 | Industry | Water |
| chloride | | | | | |
| Calcium | 471-34-1 | 1 | 0 | Others | Water |
| carbonate | | | | | |
| Carbamazepine | 298-46-4 | 0 | 1 | Pharma- | DMSO |
| | | | | ceuticals | |
| Ciprofloxacin | 86393-32-0 | 0 | 0 | Pharma- | Water |
| hydrochloride | | | | ceuticals | |
| Cyclophos- | 50-18-0 | 0 | 1 | Pharma- | Water |
| phamide | | | | ceuticals | |
| Diacetyl | 431-03-8 | 0 | 1 | Food | DMSO |
| Dipropylene | 25265-71-8 | 0 | 0 | Industry | Water |
| glycol | | | | | |
| D-sorbitol | 50-70-4 | 0 | 0 | Food | Water |
| Ethylene glycol | 107-21-1 | 0 | 0 | Industry | Water |
| Gallium (III) | 12024-21-4 | 1 | 0 | Industry | Water |
| oxide | | | | | |
| Glass beads | — | 1 | 0 | Others | Water |
| Hydroxypropyl- | 128446-35-5 | 0 | 0 | Others | DMSO |
| β-cyclodextrin | | | | | |
| 4-Ipomeanol | 32954-58-8 | 0 | 0 | Food | DMSO |
| Iron (III) oxide | 1309-37-2 | 1 | 0 | Industry | Water |
| Ketoconazole | 65277-42-1 | 0 | 0 | Pharma- | DMSO |
| | | | | ceuticals | |
| Lincomycin | 859-18-7 | 0 | 0 | Pharma- | Water |
| hydrochloride | | | | ceuticals | |
| Lithium | 7447-41-8 | 0 | 0 | Industry | Water |
| chloride | | | | | |
| Melatonin | 73-31-4 | 0 | 0 | Pharma- | DMSO |
| | | | | ceuticals | |
| Methotrexate | 59-05-2 | 0 | 1 | Pharma- | DMSO |
| | | | | ceuticals | |
| Monocrotaline | 315-22-0 | 0 | 0 | Food | EtOH |
| β-Myrcene | 123-35-3 | 0 | 0 | Personal | DMSO |
| | | | | care | |
| Naltrexone | 16676-29-2 | 0 | 0 | Pharma- | Water |
| hydrochloride | | | | ceuticals | |
| Nevirapine | 129618-40-2 | 0 | 0 | Pharma- | DMSO |
| | | | | ceuticals | |
| Nickel sulfate | 10101-97-0 | 0 | 1 | Industry | Water |
| Nitrofurantoin | 67-20-9 | 0 | 1 | Pharma- | DMSO |
| | | | | ceuticals | |
| NNK | 64091-91-4 | 0 | 1 | Others | Water |
| Nystatin | 1400-61-9 | 0 | 0 | Pharma- | DMSO |
| | | | | ceuticals | |
| Paraquat | 1910-42-5 | 0 | 1 | Others | Water |
| Phenacetin | 62-44-2 | 0 | 0 | Pharma- | DMSO |
| | | | | ceuticals | |
| p-Phenylene- | 106-50-3 | 0 | 1 | Personal | Water |
| diamine | | | | care | |
| Quartz | 14808-60-7 | 1 | 1 | Others | Water |
| Rutile | 1317-80-2 | 1 | 1 | Others | Water |
| Silica | 112945-52-5 | 1 | 1 | Personal | Water |
| | | | | care | |
| Sodium | 7647-14-5 | 0 | 0 | Food | Water |
| chloride | | | | | |
| Temsirolimus | 162635-04-3 | 0 | 1 | Pharma- | DMSO |
| | | | | ceuticals | |
| Tenofovir | 147127-20-6 | 0 | 0 | Others | DMSO |
| Thiamethoxam | 153719-23-4 | 0 | 0 | Others | DMSO |
| Titanium | 13463-67-7 | 1 | 1 | Personal | Water |
| dioxide | | | | care | |
| Triethylene | 112-27-6 | 0 | 0 | Industry | Water |
| glycol | | | | | |
| Triiodo- | 6893-02-3 | 0 | 0 | Pharma- | DMSO |
| thyronine | | | | ceuticals | |
| Vancomycin | 1404-93-9 | 0 | 0 | Pharma- | Water |
| hydrochloride | | | | ceuticals | |
| Vinylidene | 75-35-4 | 0 | 0 | Industry | DMSO |
| chloride | | | | | |

### Cell Culture and Compound Treatment

We used two commercially available immortalized cell lines A549 (CCL-185™) and BEAS-2B (CRL-9609™) from the American Type Culture Collection (ATCC). A549 was cultured in Roswell Park Memorial Institute (RPMI) 1640 medium (Gibco) supplemented with 10% fetal bovine serum (HyClone™) and 1% penicillin/streptomycin (Gibco). BEAS-2B was maintained in Bronchial Epithelial Cell Growth Medium (BEGM) (Lonza/Clonetics™) and 1% penicillin/streptomycin (Gibco); all supplement provided in BEGM Bullet Kit was used except GA-1000 (gentamycin-amphotericin B mix). Only passages before P15 of A549 and BEAS-2B were used in this study.

Cells were seeded into 384-well black plates with transparent coverglass bottom (Nunc). All cells were cultured for 48 hours before overnight treatment with respective compounds (16 hours). The concentration of the tested compounds were 31.3, 62.5, 125, 250, 500, 1000, 2000 μM for soluble and 16.13, 31.3, 62.5, 125, 250, 500, 1000 μg/mL for particulate compounds. Positive, negative, and vehicle controls (DMSO, ethanol or water, depending on the solvent of the tested compounds) and four technical replicates were performed for each compound and dosage.

### Immunostaining

After compound treatment for 16 hours, cells were fixed using 4% paraformaldehyde in phosphate-buffered saline (PBS). The cells were permeabilized with tris-buffered saline with 0.1% triton-X (TBST) and blocked for 1 hour with TBST containing 5% bovine serum albumin (BSA). The samples were incubated with a rabbit monoclonal antibody to γH2AX (phospho S139) (Cell Signaling Technology) at 1:500 at 4° C. overnight. Subsequently, the cells were incubated with a goat anti-rabbit secondary antibody conjugated to Alexa488 (Invitrogen) and HCS CellMask™ deep red at 1:500 and 1:2000 respectively for about 1 hour. Finally, the cells were stained with Hoechst 33342 (Invitrogen) at 1:800 and DyLight™ 554 phalloidin (Cell Signaling Technology).

### Image Acquisition

Imaging was performed with a 20× objective using the Zeiss Axio Observer Z1 system with definite laser focus (Zeiss). Four different channels were used to image blue (DAPI), green (488), red (DsRed), and far-red (Cy5) fluorescence. Four sites per well were imaged. The images were saved in 16-bit TIFF format.

### Automated Cellular Phenotypic Profiling

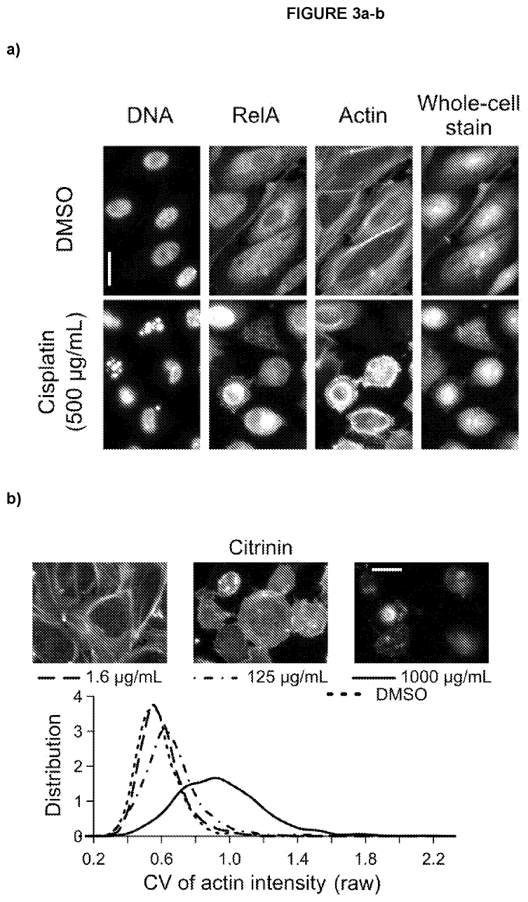

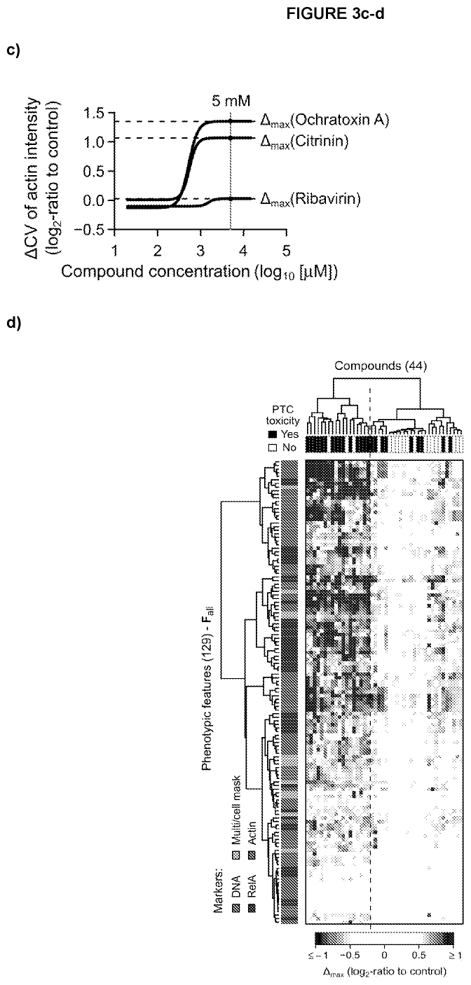

Our phenotypic profiling strategy (FIG. 2) was to automatically measure a large numbers of quantitative phenotypic features of primary human PTCs under in vitro conditions, and then systematically screen for a subset of phenotypic features that were the most predictive of in vivo PTC toxicity. Our features were based on four fluorescent markers (FIG. 3a). We used 4′,6-diamidino-2-phenylindole (DAPI) and rhodamine phalloidin to label the DNA and actin cytoskeleton, respectively. Nuclear and chromatin structure alterations are involved in many fundamental cellular processes, such as transcription, mitosis, and cell death. Actin filaments play an important role in maintaining the cellular function of PTCs (Kellerman et al., Am J Physiol 259:F279-285 (1990)). We also labeled the cells with an antibody specific for a subunit of the nuclear factor (NF)-κB complex, RelA. This was motivated by our previous models based on IL-6/8 (Li et al., Toxicol Res 2: 352-365 (2013); Li et al., Mol Pharm 11: 1982-1990 (2014); Su et al., BMC Bioinformatics 15: S16 (2014); Kandasamy et al., Sci Rep. doi: 10.1038/srep12337 (2015)), which are regulated by the NF-κB complex (Matsusaka et al., Proc Natl Acad Sci 90: 10193-10197 (1993)). The final marker was a whole-cell stain (WCS) used to facilitate automated cell segmentation and measurements of cellular morphology features.

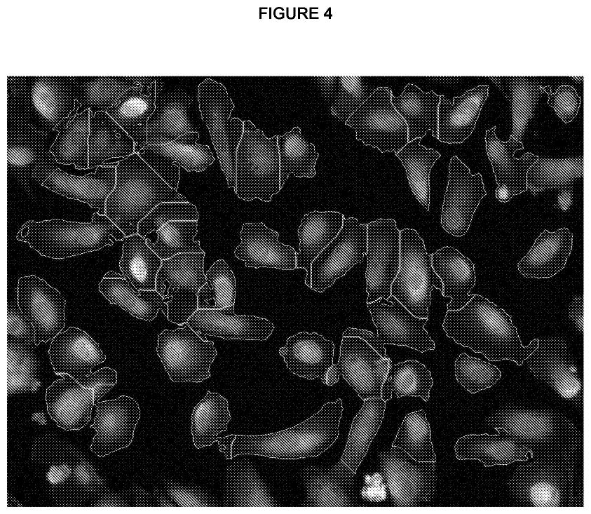

After compound treatment, we stained PTCs with these four fluorescent markers and imaged them using a high-throughput imaging system. We automatically identified ˜500-1000 cells from 36 microscopy images captured for each compound and treatment dosage (FIG. 4). Then, for each cell, we extracted 129 quantitative phenotypic features (FIG. 3b), which include 78 Haralick's texture features (Haralick et al., IEEE Trans Syst Man Cybern SMC-3: 610-621 (1973)) (measuring the statistics of the spatial co-occurrence patterns of the markers), 29 intensity features (measuring the staining levels of the markers at different subcellular regions), 9 intensity-ratio features (measuring the ratios between intensity features), 6 correlation features (measuring the spatial correlations between two markers at the single-cell level), and 6 morphology features (measuring the shape properties of the nuclear and cellular regions). We also included cell count as a feature. Similar phenotypic features and profiling methods were previously used to classify large numbers of small molecules according to their targets/mechanisms (Loo et al., Nat Methods 4: 445-453 (2007)). Therefore, we hypothesized that a subset of these features might also be discriminative enough to predict PTC toxicity.

For each phenotypic feature, we first computed the log₂-ratios (“Δ”) of its values at all the tested dosages with respect to the vehicle controls. Then, we estimated the feature's dose response curve (DRC) using a standard log-logistic model, and its maximum response value (“Δₘₐₓ”) from the curve (FIG. 3c). Finally, the median Δₘₐₓ values across three biological replicates were computed. The final dataset (“HPTC-A”) was a matrix of Δₘₐₓ values for 129 phenotypic features (Fₐₗₗ={f₁, f₂, . . . , f₁₂₉} rows) rows) and 44 xenobiotic compounds (columns) (FIG. 3d). For brevity, all features that we mention in this article are referring to the Δₘₐₓ parameters of the respective features and not the raw measured feature values, unless otherwise indicated. Hierarchical clustering of the compounds based on the phenotypic feature values revealed two major clusters (FIG. 3d). One of them was significantly enriched with the PTC-toxic compounds (83% of the cluster were PTC-toxic compounds; P=1.59×10⁻³, hypergeometric test), and the other one was significantly enriched with the non-PTC-toxic compounds (65% of the cluster were non-PTC-toxic compounds; P=1.59×10⁻³, hypergeometric test). Most of the phenotypic features showed larger changes under the first cluster than the second cluster, suggesting that non-PTC-toxic compounds only induced small or no change in the primary human PTCs. We also performed similar clustering analysis on the phenotypic features, and found two major clusters corresponding to either increased or decreased feature values after treatments with PTC-toxic compounds (FIG. 3d). Features from all markers were represented in both clusters. Therefore, most of our phenotypic features are diverse and capture both increasing and decreasing properties of the markers.

### Nuclear and Cytoskeletal Features are Highly Predictive

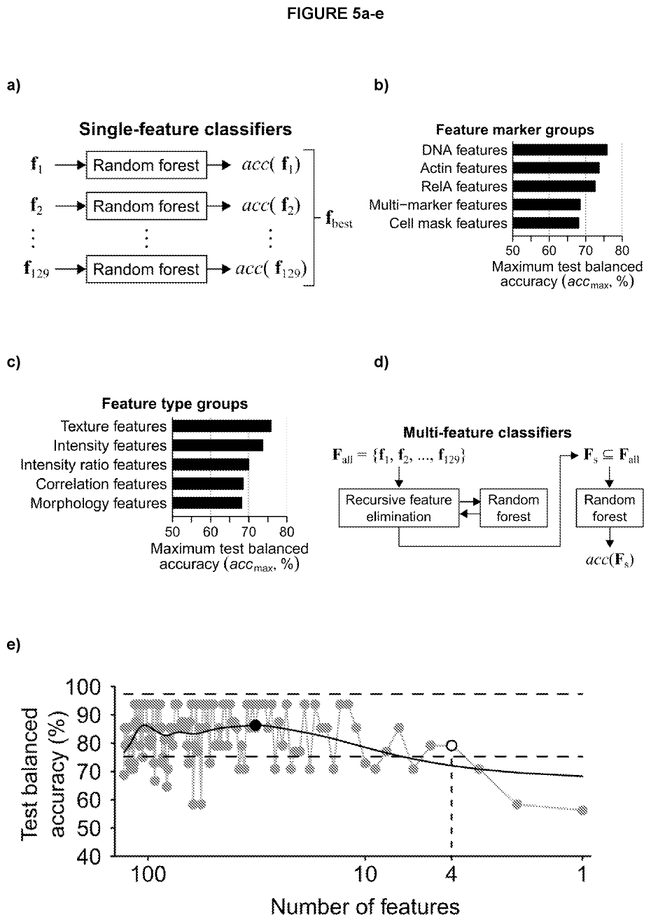

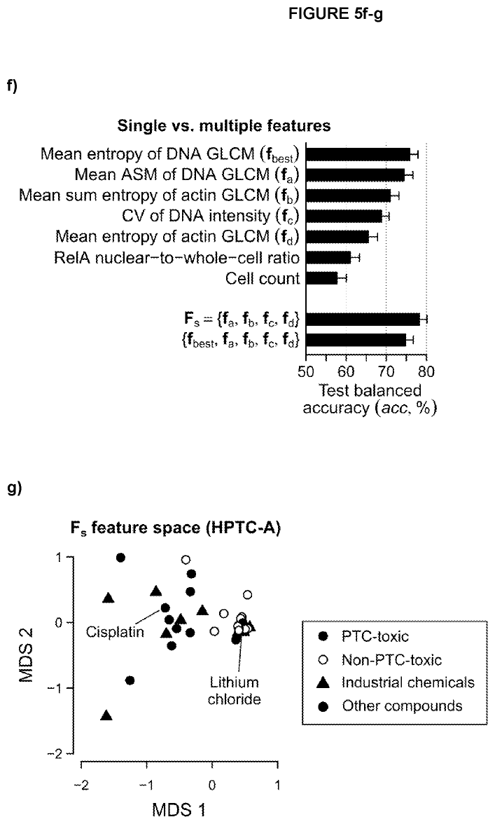

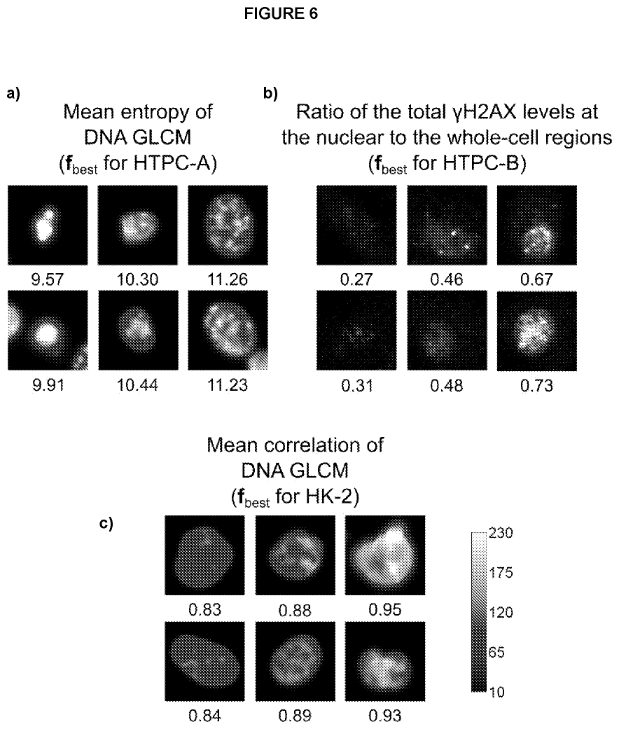

To test each individual feature, we constructed a binary classifier based on the feature using a random forest algorithm (Breiman Mach Learn 45: 5-32 (2001); Su et al., BMC Bioinformatics 15: S16 (2014)), and estimated the prediction accuracy using a 10-fold cross validation procedure (FIG. 5a and Methods). The balanced accuracy (average of sensitivity and specificity) of a binary classifier ranges from 50% (performance of a trivial random classifier) to 100% (maximum). The training accuracy is the accuracy in classifying the training data used to build the classifier, and the test accuracy is the accuracy in classifying independent test data unused during the training process. Our evaluation procedure ensured that the training and test data were statistically independent. In our unbiased approach for phenotypic profiling, we started with a large number of general phenotypic features, but only expected a small number of features to be discriminative. Therefore, when comparing different groups of features, we only considered the maximum accuracy of a feature group (which was based on the best single feature within the group) and not the average accuracy of the group. For the HPTC-A dataset, we found that all single features had ˜97% and above training accuracy, indicating most training samples could be separated according to their known in vivo PTC toxicity. The feature marker group with the highest maximum test accuracy was DNA (75.8%), followed by actin (73.7%) and RelA (72.6%) (FIG. 5b). Surprisingly, RelA features were not highly predictive of PTC toxicity. For example, the RelA nuclear-to-whole-cell intensity ratio, which is an indicator of NF-κB nuclear translocation and transcriptional activation of its downstream effectors (Deptala et al., Cytometry 33: 376-382 (1998)), had 98.8% training but only 61.0% test accuracies. The feature type groups with the highest maximum test accuracy was Haralick's texture (Haralick et al., IEEE Trans Syst Man Cybern SMC-3: 610-621 (1973)) (75.8%), followed by intensity (73.7%) and intensity ratio (69.9%) (FIG. 5c). In fact, six of the ten best-performing single features were all Haralick's texture features, which are based on the grey-level co-occurrence matrices (GLCM) (Haralick et al., IEEE Trans Syst Man Cybern SMC-3: 610-621 (1973)) of the fluorescent markers. The GLCM of a marker summarizes the distribution of spatial transitions between different intensity levels of the marker in a cell image (Haralick et al., IEEE Trans Syst Man Cybern SMC-3: 610-621 (1973)). Haralick's features, which describe various statistical properties of a GLCM, can be used to represent the textural patterns found in the image (Methods). The best single feature, f_{best}, among all the 129 features was the “mean entropy” of the DNA GLCM (99.3% training and 75.8% test accuracies). The feature is a measure of the homogeneity of the DNA GLCM. Cell images with more “random” DNA staining patterns, where the transitions between all intensity levels are more equally probable, have more homogenous GLCMs and thus higher values of GLCM entropy (FIG. 6). Overall, changes in the texture of the DNA and actin cytoskeleton localization patterns were highly predictive of the in vivo PTC toxicity of xenobiotics with diverse chemical structures. The high accuracy underscores the importance and advantage of using image-based phenotypic features as in vitro toxicity endpoints.

Multiple Features are More Predictive than Single Features

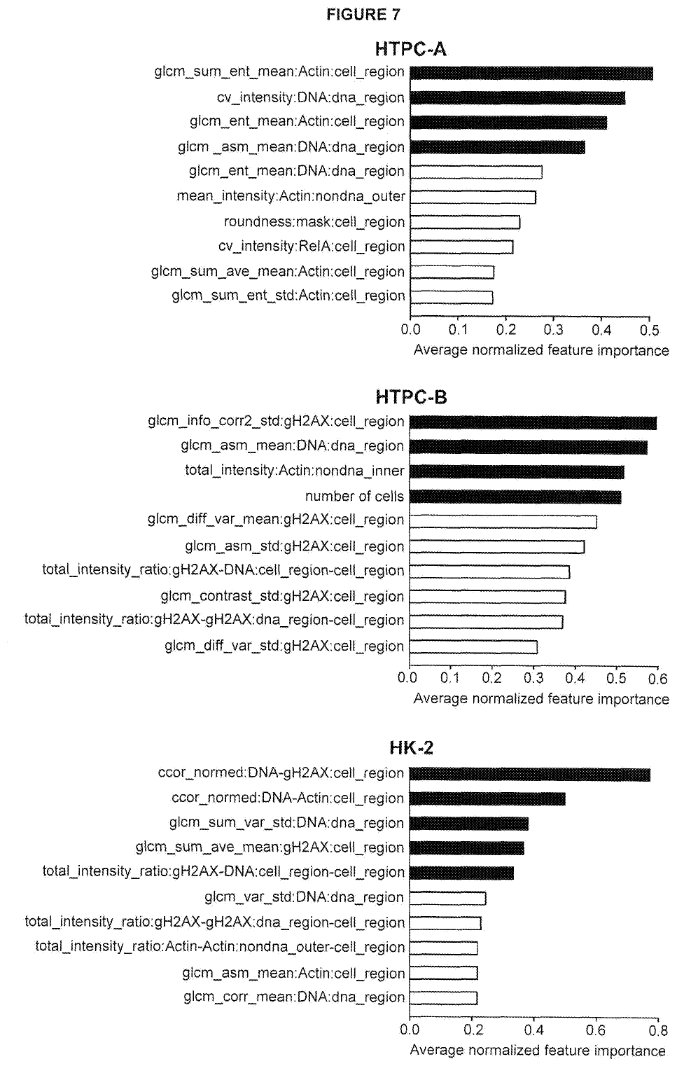

Xenobiotic compounds may induce different types of PTC injuries and responses. Therefore, classifiers based on multiple different phenotypic endpoints are more likely to give higher overall prediction accuracy. To preserve the dependency between features, we trained multi-dimensional classifiers based on multiple features simultaneously (FIG. 5d). Then, a recursive feature elimination algorithm (Loo et al., Nat Methods 4: 445-453 (2007)) was used to automatically remove irrelevant and/or redundant features (FIG. 5e and Methods). The number of retained features was automatically determined based on the training data only. Therefore, the process was repeated for every cross-validation fold, which had different training data. The features were ranked according to their importance values, which were estimated by the random forest algorithm (Breiman Mach Learn 45: 5-32 (2001)) and averaged across all the cross validation folds. For the HPTC-A dataset, we found a set of four features (Fₛ) that had the highest average importance values (FIG. 7). These features were the “coefficient of variation (CV)” of DNA intensity at the nuclear region, “mean angular second moment (ASM)” of the DNA GLCM, “mean sum of entropy” and “mean entropy” of the actin GLCM (FIG. 5f and Table 3).

TABLE 3
Summary of overall nephrotoxicity prediction performances for
single- and multi-feature classifiers for all three data sets.

| | | Com- | | | Balanced | | | | | |
|---|---|---|---|---|---|---|---|---|---|---|
| | | pound | Feature | | accuracy | | Sensitivity | | Specificity | |
| Dataset | Markers | number | number | Feature names (cellXpress format) | Training | Test | Training | Test | Training | Test |
| HPTC-A | DNA/RelA/Actin/WCS | 44 | 1 | glcm_ent_mean:DNA:dna_region | 99.3 | 75.8 | 99.6 | 81.2 | 99.1 | 70.5 |
| HPTC-A | DNA/RelA/Actin/WCS | 44 | 4 | glcm_sum_ent_mean:Actin:cell_ | 99.5 | 78.3 | 99.4 | 76.5 | 99.7 | 80.0 |
| | | | | region, cv_intensity:DNA:dna_region, | | | | | | |
| | | | | glcm_ent_mean:Actin:cell_region, | | | | | | |
| | | | | glcm_asm_mean:DNA:dna_region | | | | | | |
| HPTC-A | DNA/RelA/Actin/WCS | 42 | 4 | glcm_sum_ent_mean:Actin:cell_ | 99.6 | 77.8 | 99.7 | 75.2 | 99.6 | 80.5 |
| | | | | region, cv_intensity:DNA:dna_region, | | | | | | |
| | | | | glcm_ent_mean:Actin:cell_region, | | | | | | |
| | | | | glcm_asm_mean:DNA:dna_reaion | | | | | | |
| HPTC-B | DNA/γH2AX/Actin/WCS | 42 | 1 | total_intensity_ratio:gH2AX- | 99.5 | 77.6 | 99.4 | 72.2 | 99.7 | 83.0 |
| | | | | gH2AX:dna_region-cell_region | | | | | | |
| HPTC-B | DNA/γH2AX/Actin/WCS | 42 | 4 | total_intensity:Actin:nondna_inner, | 99.7 | 81.6 | 99.9 | 83.7 | 99.6 | 79.5 |
| | | | | glcm_asm_mean:DNA:dna_region, | | | | | | |
| | | | | glcm_info_corr2_std:gH2AX:cell_ | | | | | | |
| | | | | region, cellcount | | | | | | |
| HK-2 | DNA/γH2AX/Actin/WCS | 42 | 1 | glcm_corr_mean:DNA:dna_region | 99.8 | 83.9 | 99.6 | 78.3 | 99.9 | 89.5 |
| HK-2 | DNA/γH2AX/Actin/WCS | 42 | 5 | ccorr_normed:DNA-gH2AX:cell_region, | 99.9 | 88.9 | 100.0 | 98.8 | 99.8 | 79.0 |
| | | | | ccorr_normed:DNA-Actin:cell_region, | | | | | | |
| | | | | glcm_sum_ave_mean:gH2AX:cell_ | | | | | | |
| | | | | region, total_intensity_ratio:gH2AX- | | | | | | |
| | | | | DNA:cell_region-cell_region, | | | | | | |
| | | | | glcm_sum_var_std:DNA:dna_region | | | | | | |

| Feature names (cellXpress format) | Descriptions |
|---|---|
| ccorr_normed:DNA-gH2AX:cell_region | Normalized spatial correlation coefficient between DNA and γH2AX |
| | intensities at the whole-cell region |
| ccorr_normed:DNA-Actin:cell_region | Normalized spatial correlation coefficient between DNA and actin intensities |
| | at the whole-cell region |
| cellcount | Cell count |
| cv_intensity:DNA:dna_region | Coefficient of variation (CV) of the DNA intensity at the nuclear region |
| glcm_asm_mean:DNA:dna_region | Mean angular second moment (ASM) of DNA GLCM at the nuclear region |
| glcm_corr_mean:DNA:dna_region | Mean correlation of DNA GLCM at the nuclear region |
| glcm_ent_mean:Actin:cell_region | Mean entropy of the actin GLCM at the whole-cell region |
| glcm_ent_mean:DNA:dna_region | Mean entropy of the DNA GLCM at the nuclear region |
| glcm_info_corr2_std:rH2AX:cell_region | Standard deviation of the information measure of correlation 2 of γH2AX |
| | GLCM at the whole-cell region |
| glcm_sum_ave_mean:gH2AX:cell_region | Mean sum average of γH2AX GLCM at the whole-cell region |
| glcm_sum_ent_mean:Actin:cell_region | Mean sum entropy of the actin GLCM at the whole-cell region |
| glcm_sum_var_std:DNA:dna_region | Standard deviation of the sum variance of DNA GLCM at the nuclear region |
| total_intensity:Actin:nondna_inner | Total actin intensity level at the inner cytoplasmic region |
| total_intensity_ratio:gH2AX- | Ratio of the total γH2AX to DNA intensities at the whole-cell region |
| DNA:cell_region-cell_region | |
| total_intensity_ratio:gH2AX- | Ratio of the total γH2AX intensity levels at the nuclear region to the whole- |
| gH2AX:dna_region-cell_region | cell region |

Similar to the single-feature classification results, these top features were all based on the DNA and cytoskeleton markers, and three of them were texture features. We trained multi-feature classifiers using these four features and obtained 99.5% training and 78.3% test accuracies, which were higher than the performances of all single-feature classifiers (FIG. 5f). The individual test accuracies of these four features only ranged between 65.5-74.4%. Therefore, combining the features together increased the prediction performance. We also found that the inclusion of f_{best} into Fₛ did not further increase the prediction accuracy of our models (FIG. 5f), indicating our recursive feature elimination algorithm was highly effective.

In the feature space of Fₛ, we found that most of the non-toxic compounds formed a tight cluster, where they were closer to each other than to the toxic compounds (FIG. 5g). Six of the eight toxic industrial chemicals with simple chemical structures could now be clearly separated from the non-toxic compounds (FIG. 5g). This separation was not evident in the original chemical structure space (FIG. 1b). Among all the tested compounds, 22 compounds had consistently 100% average test accuracy in both the single- and multi-feature classifiers (Table 4).

TABLE 4
Average nephrotoxicity test accuracies for individual compounds.

| | | HPTC- | HPTC- | HPTC- | HPTC- | | |
|---|---|---|---|---|---|---|---|
| | PTC | A | A | B | B | HK-2 | HK-2 |
| Drug name | Toxicity | (Single) | (Multi) | (Single) | (Multi) | (Single) | (Multi) |
| Lead(IV) acetate | Toxic | 0% | 80% | NA | NA | NA | NA |
| Aristolochic acid | Toxic | 100% | 100% | 100% | 100% | 100% | 100% |
| Arsenic(III) oxide | Toxic | 100% | 100% | 100% | 100% | 100% | 100% |
| Cadmium(II) chloride | Toxic | 100% | 100% | 100% | 100% | 100% | 100% |
| Cephalosporin C | Toxic | 100% | 100% | 100% | 100% | 100% | 100% |
| Cephalothin | Toxic | 100% | 100% | 100% | 100% | 100% | 100% |
| Citrinin | Toxic | 100% | 100% | 100% | 100% | 100% | 100% |
| Gold(I) chloride | Toxic | 100% | 100% | 100% | 100% | 100% | 100% |
| Tacrolimus | Toxic | 10% | 100% | 100% | 100% | 100% | 100% |
| Copper(II) chloride | Toxic | 0% | 20% | 100% | 100% | 100% | 100% |
| Rifampicin | Toxic | 0% | 100% | 90% | 100% | 100% | 100% |
| Puromycin | Toxic | 90% | 0% | 0% | 100% | 100% | 100% |
| Cephaloridine | Toxic | 100% | 0% | 20% | 90% | 100% | 100% |
| Bismuth(III) oxide | Toxic | 100% | 100% | 0% | 90% | 100% | 100% |
| Tenofovir | Toxic | 80% | 0% | 100% | 20% | 100% | 100% |
| Cisplatin | Toxic | 100% | 100% | 100% | 0% | 100% | 100% |
| Gentamicin | Toxic | 90% | 0% | 0% | 100% | 90% | 100% |
| Cyclosporin A | Toxic | 100% | 40% | 0% | 80% | 90% | 100% |
| Ochratoxin A | Toxic | 100% | 100% | 100% | 100% | 20% | 100% |
| Tetracycline | Toxic | 100% | 100% | 100% | 100% | 0% | 100% |
| Paraquat | Toxic | 90% | 100% | 100% | 100% | 0% | 100% |
| Potassium dichromate | Toxic | 100% | 100% | 100% | 80% | 100% | 90% |
| 5-Fluorouracil | Toxic | 100% | 100% | 20% | 40% | 0% | 90% |
| Germanium(IV) oxide | Toxic | 100% | 90% | 0% | 10% | 0% | 90% |
| Hydrocortisone | Non-toxic | 30% | 90% | NA | NA | NA | NA |
| Acarbose | Non-toxic | 100% | 100% | 100% | 100% | 100% | 100% |
| Ethylene glycol | Non-toxic | 100% | 100% | 100% | 100% | 100% | 100% |
| Glycine | Non-toxic | 100% | 100% | 100% | 100% | 100% | 100% |
| Lincomycin | Non-toxic | 100% | 100% | 100% | 100% | 100% | 100% |
| Lindane | Non-toxic | 100% | 100% | 100% | 100% | 100% | 100% |
| Phenacetin | Non-toxic | 100% | 100% | 100% | 100% | 100% | 100% |
| Furosemide | Non-toxic | 0% | 100% | 100% | 100% | 100% | 100% |
| Dexamethasone | Non-toxic | 100% | 90% | 100% | 100% | 100% | 100% |
| Valacyclovir | Non-toxic | 100% | 90% | 100% | 100% | 100% | 100% |
| Metformin | | | | | | | |
| hydrochloride | Non-toxic | 90% | 50% | 100% | 100% | 100% | 100% |
| Melatonin | Non-toxic | 100% | 90% | 10% | 100% | 100% | 100% |
| Lithium chloride | Non-toxic | 0% | 90% | 100% | 90% | 100% | 100% |
| Ciprofloxacin | Non-toxic | 0% | 0% | 0% | 0% | 100% | 100% |
| Ibuprofen | Non-toxic | 100% | 100% | 100% | 100% | 0% | 100% |
| Triiodothyronine | Non-toxic | 100% | 100% | 100% | 100% | 0% | 90% |
| Levodopa | Non-toxic | 0% | 0% | 30% | 10% | 100% | 0% |
| Vancomycin | Non-toxic | 100% | 100% | 100% | 0% | 100% | 0% |
| Ribavirin | Non-toxic | 90% | 0% | 30% | 0% | 100% | 0% |
| Acetaminophen | Non-toxic | 0% | 100% | 100% | 100% | 90% | 0% |

Only three compounds, namely ciprofloxacin (antibiotic), levodopa (psychoactive drug), and copper(II) chloride (industrial chemical), had consistently <50% test accuracy in both types of classifiers. These results show that our computational models were general and did not favor specific classes of compounds.

### The Most Important Feature Indicates Induction of a DNA Damage Response

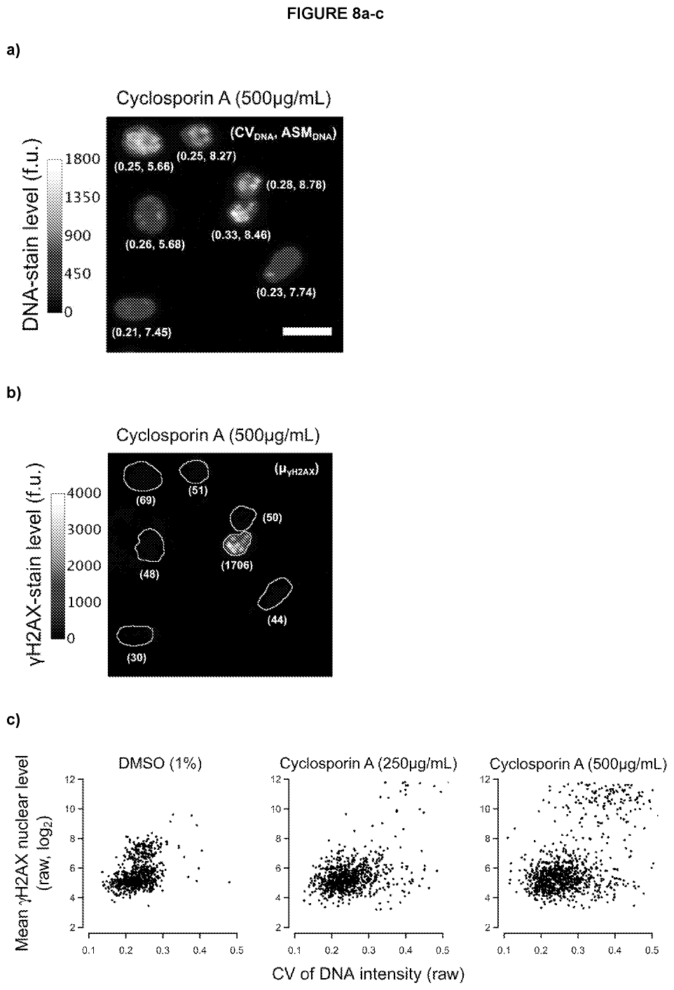

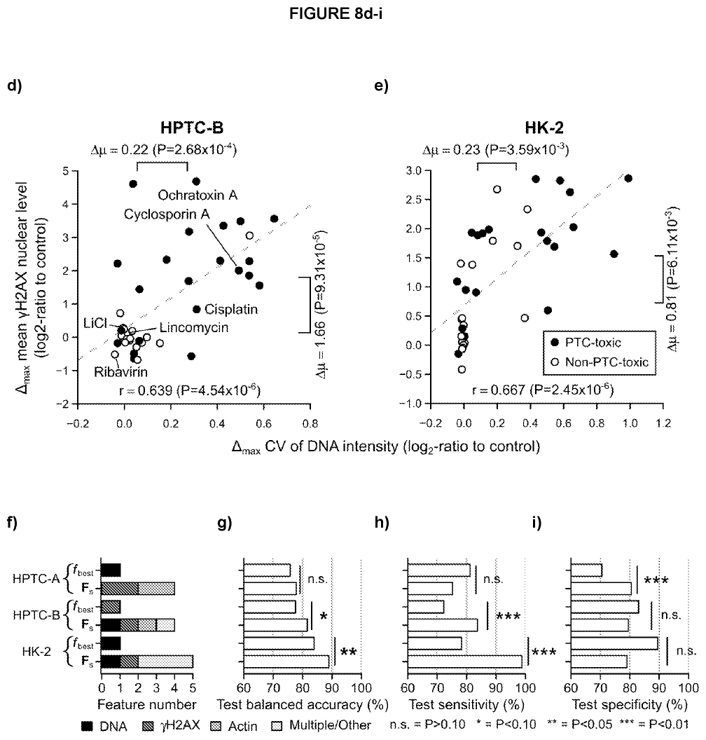

To further investigate the type of cell injury and damage response represented by our phenotypic features, we focused on the two DNA features in Fₛ, namely 1) the mean ASM of the DNA GLCM, which had the highest single-feature test accuracy among the four selected features, and 2) the CV of DNA intensity at the nuclear region (FIG. 5f). ASM is a measure of the heterogeneity of a DNA GLCM (Methods and FIG. 8a). The feature gives high values when the transitions between certain intensity levels are dominant (for example, when the intensity values form certain regular shapes), or low values when all transitions are equally probable (for example, when the intensity values are diffused and randomly distributed). CV, which is equal to standard deviation divided by mean, is a standardized measure of the dispersion of a set of values, which in our case were the DNA staining intensity levels within the nuclear region. By examining the fluorescence microscopy images of the cells, we found that cells with high values of these two features had disconnected, highly irregular, and punctate DNA staining levels (FIG. 8a), indicating profound changes in chromatin structure and the formation of distinct chromatin domains. However, the overall nuclear and cellular morphologies of these cells were still remained largely intact and not fragmented, as in typical apoptotic cells. Therefore, we hypothesized that the features may indicate a DNA damage response, which is known to be associated with the formation of distinct chromatin domains in the megabase size range and large-scale chromatin reorganization (Rogakou et al., J Biol Chem 273: 5858-5868 (1998); Jakob et al., Nucleic Acids Res 39: 6489-6499 (2011)).

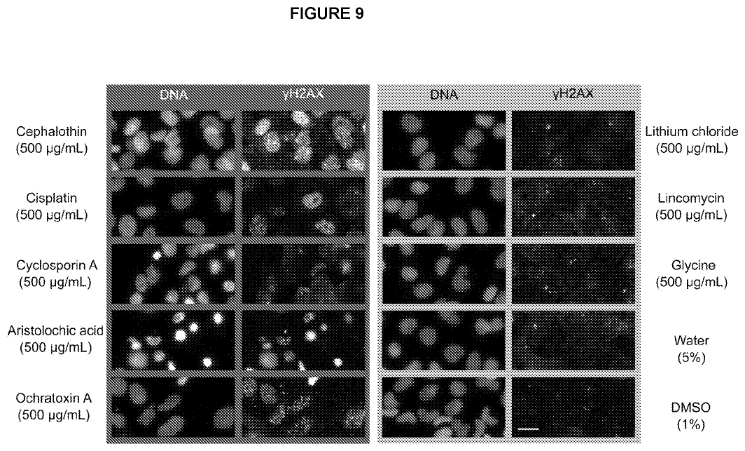

To test our hypothesis, we repeated the treatment experiments for 42 reference compounds, but replacing the RelA marker with an antibody specific for histone H2AX phosphorylated on serine 139 (γH2AX), which is a DNA damage response marker (Rogakou et al., J Biol Chem 273: 5858-5868 (1998)) (FIG. 8b). Under endogenous or exogenous DNA damage conditions, γH2AX is induced and recruits repair factors to the sites of double-strand breaks (Paull et al., Curr Biol 10: 886-895 (2000)). We repeated the experiments in both primary human PTCs (the “HPTC-B” dataset) and an immortalized human PT cell line, human kidney 2 (the “HK-2” dataset, FIG. 9). At the single-cell level, we found that cells with higher raw DNA CV levels induced by xenobiotic compounds tended to have higher raw mean γH2AX nuclear levels, but the responses might be highly heterogeneous (FIG. 8b). For example, 500 μg/mL cyclosporine A caused ˜40-fold increases in the raw mean γH2AX nuclear levels, but only in ˜13% of the cells (FIG. 8c). Nevertheless, due to the large increases in magnitude, the effects could still be detected at the population-averaged level. Similar increases in γH2AX nuclear levels were also observed in cells treated with other PTC-toxic compounds (FIG. 9). Across all the tested compounds, the maximum increases (i.e. Δₘₐₓ) in DNA CV and mean nuclear γH2AX levels were strongly and positively correlated to each other in both primary and HK-2 cells (r=0.639 and 0.667, respectively; FIGS. 8d and e). Furthermore, both features were significantly higher in cells treated with the PTC-toxic compounds than those with the non-PTC-toxic compounds (all P<0.01, one-tailed t-test; FIGS. 8d and e). These results suggest that most of the PTC-toxic compounds induce a DNA damage response, even though many of them are not known to bind to DNA directly.

### Improved Computational Models Based on γH2AX

To what extent can the γH2AX marker improve the prediction performance of our computational models? We repeated the same phenotypic profiling procedure but using 129 phenotypic features based on the DNA, γH2AX and actin markers (FIGS. 8f and g). For the HTPC-B dataset, we found that the best single feature f_{best} was the ratio of total γH2AX levels at the nuclear to the whole-cell regions (99.5% training and 77.6% test accuracies), which indicates the activation of γH2AX at the nuclear region (FIG. 6b). The best multi-feature set Fₛ were four nuclear and actin cytoskeletal features (99.7% training and 81.6% test accuracies, see FIG. 7 and Table 2 for the complete listing of features). For the HK-2 dataset, we found that its f_{best} was the mean correlation of DNA GLCM (99.8% training and 83.9% test accuracies), which is a measure of the linear dependency of intensity levels of neighboring pixels (FIG. 6c). The best multi-feature set Fₛ were five chromosomal and cytoskeleton features (99.9% training and 88.9% test accuracies, FIG. 7 and Table 2). For both datasets (HPTC-B and HK-2), we found a consistent trend that multi-feature classifiers had higher test accuracies than single-feature classifiers (FIGS. 8f and g). However, single-feature classifiers had higher test specificities, while multi-feature classifiers had higher sensitivities (FIGS. 8h and i). Furthermore, the number of compounds that could be predicted with 100% average test accuracy in both single- and multi-feature classifiers had increased from 22 (HPTC-A) to 25 (HPTC-B) or 28 (HK-2) (Table 4). Together, these results show that the inclusion of the γH2AX marker allowed us to obtain higher prediction accuracies.

We also compared the optimum phenotypic features selected for all three datasets (Table 3), and found several interesting and consistent trends. First, the mean ASM of DNA GLCM was automatically selected in the Fₛ's for both HPTC-A and -B datasets. Second, the f_{best} for the HPTC-B dataset (i.e., ratio of total γH2AX levels at the nuclear to the whole-cell regions) and one of the features in the Fₛ for the HK-2 dataset (i.e., ratio of total γH2AX and DNA intensity levels at the whole-cell region) are two closely-related features that indicate nuclear increase of γH2AX levels (FIG. 6). Third, although the best single features were based on DNA or γH2AX markers, actin features were still retained by the multi-feature classifiers, suggesting that the actin marker was needed to correctly classify some compounds that induced actin remodeling. Together, these results show that our predictive models are highly reproducible, and a xenobiotic-induced DNA damage response is a general phenomenon that occurs in both human primary and immortalized PTCs.

### Cell Death Responses are Variable

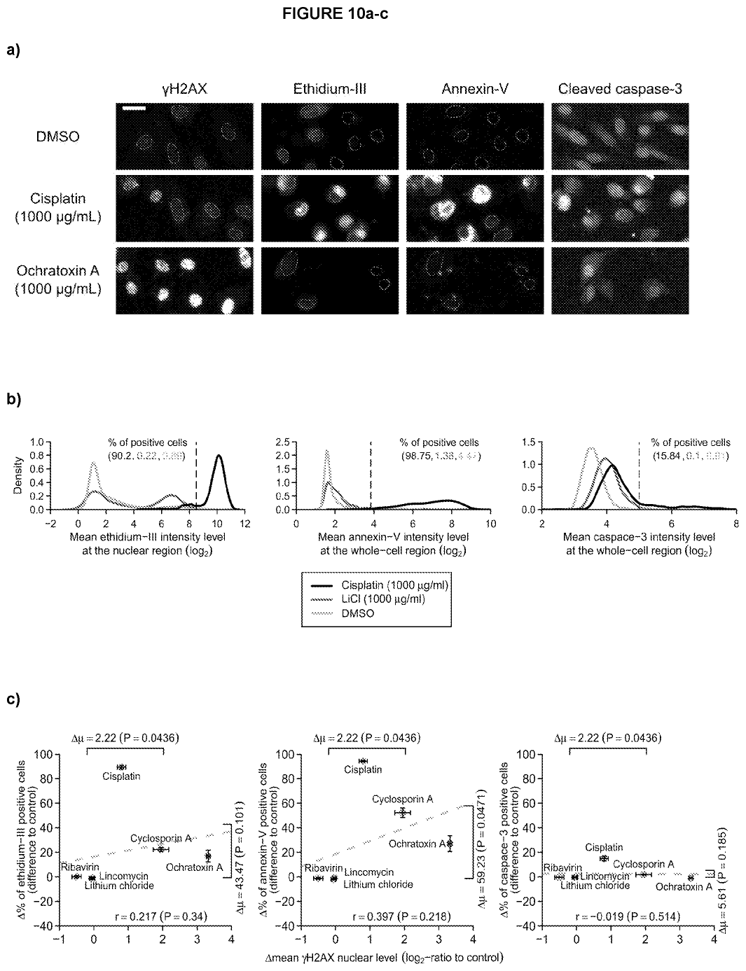

Is the DNA damage response associated with cell death under in vitro conditions? Based on the HPTC-B results, we selected three PTC-toxic compounds (cisplatin, cyclosporin A, and ochratoxin A) that induced increasing levels of γH2AX, and three non-PTC-toxic compounds (ribavirin, lithium chloride, and lincomycin) that caused small or no change in γH2AX levels (FIG. 8d). We treated primary PTCs with 1000 μg/mL of these compounds, and labeled the cells with three different cell death markers: annexin-V (a marker for the externalization of phosphatidylserine, which occurs in both apoptotic and necrotic cells (Sawai and Domae, Biochem Biophys Res Commun 411: 569-573 (2011)), cleaved caspase-3 (a marker for the activation of caspase-3, which occurs only in apoptotic cells), and ethidium homodimer III (a DNA marker that is only permeant to late apoptotic or necrotic cells due to membrane disintegration) (FIG. 10a). For each marker, we determined the percentages of positive cells under the treatments of these six compounds and solvent controls (FIG. 10b). Based on the HPTC-B dataset, we also determined the mean γH2AX nuclear levels of primary PTCs treated with 1000 μg/mL of these compounds. In agreement with our previous Δₘₐₓ measurements, the three PTC-toxic compounds induced significantly higher mean γH2AX intensity levels than the three non-PTC-toxic compounds at the tested dosage (P=0.044, FIG. 10c). However, only the increase in the percentage of annexin-V positive cells was significant (P=0.047) between the PTC-toxic and non-PTC compounds. The increases in the percentages of ethidium-homodimer-Ill and caspase-3 positive cells were less significant (both P>0.10, all one-sided t-tests; FIG. 10c). This was mostly due to the lower cell-death responses to cyclosporine A and ochratoxin A. Even for annexin-V, the responses were highly heterogeneous. For example, cyclosporine A and ochratoxin A only increased annexin-V levels in ˜50% and ˜25% of the cells, respectively. These results corroborated with our earlier results on the heterogeneity in cyclosporine A responses (FIGS. 8b and c). Surprisingly, the three PTC-toxic compounds only induced low percentages of caspase-3 positive cells (<20%). Similar lack of apoptotic responses was also previously observed for some PTC-toxic compounds, such as 5-flurouracil and gentamicin, in HK-2 cells (Wu et al., Toxicol In Vitro 23: 1170-1178 (2009)). Across all the six compounds, we found that there is no significant positive correlation between γH2AX level and these three cell death markers (all P>0.20, one-sided t-test; FIG. 10c). Together, all of these results showed that PTC toxicants induce variable cell death responses (both apoptosis and necrosis) under the tested in vitro conditions. Some of them (such as ochratoxin A, which induced a large increase in γH2AX levels) may even cause very small or no increase in cell death rates within the measured period. These results also imply that in vitro cell-death endpoints may have difficulty in accurately predicting in vivo PTC toxicity, and cannot be used to replace DNA damage features for nephrotoxicity prediction.

Pulmonary Toxicity can also be Highly Predictive Using Similar Markers

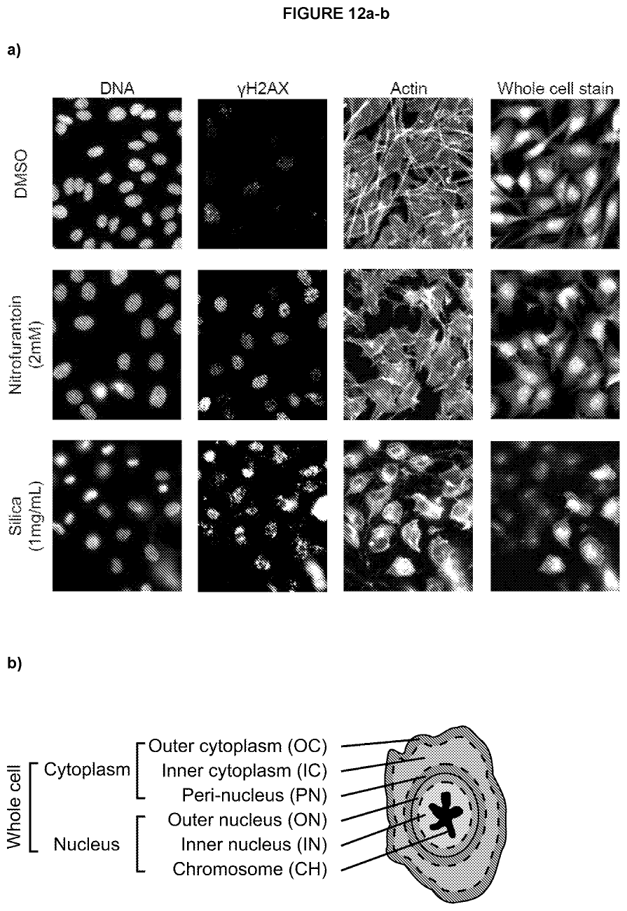

We developed in vitro pulmonary toxicity models based on immortalized alveolar cell (AVC) and bronchial epithelial cell lines (BEC). The phenotypic profiling strategy was also adopted for prediction and our features were based on four fluorescent markers and seven subcellular regions (FIGS. 12a and b). The Hoechst 33342 and DyLight™ 554 Phalloidin were used to label the DNA and actin cytoskeleton, respectively. Both nitrofurantoin and fumed silica are known to cause pulmonary diseases and silicosis respectively in humans. Immunofluorescence images show changes in the phosphorylation of a DNA damage response marker, γH2AX, and the remodeling of actin in our cell model under the treatments of DMSO (control, top), 2 mM Nitrofurantoin (middle), and 1 mg/mL fumed silica (bottom).

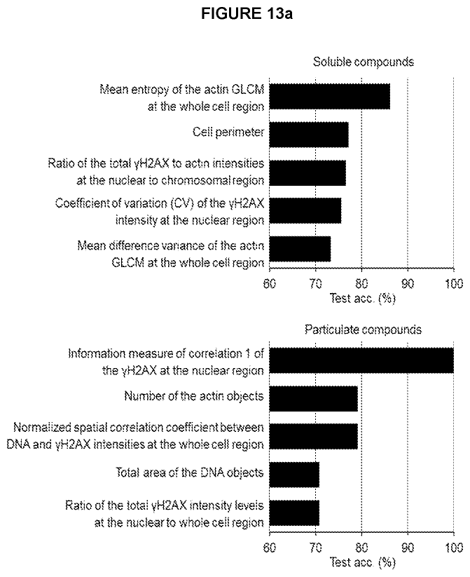

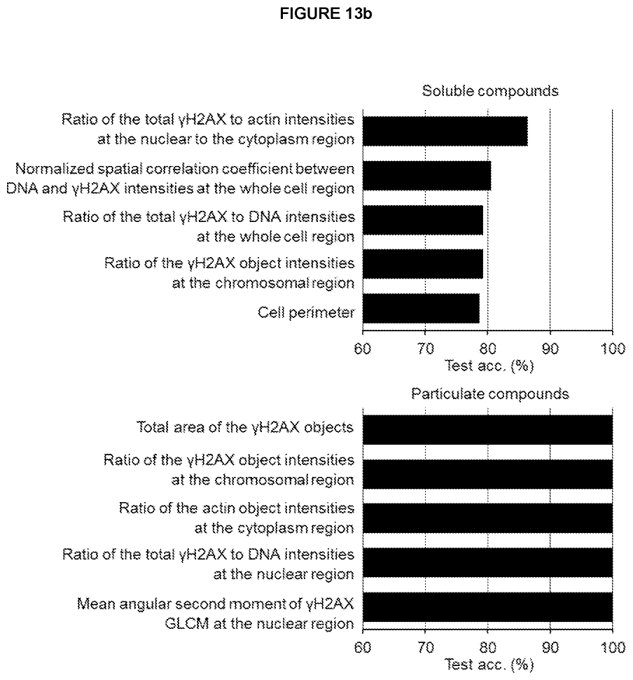

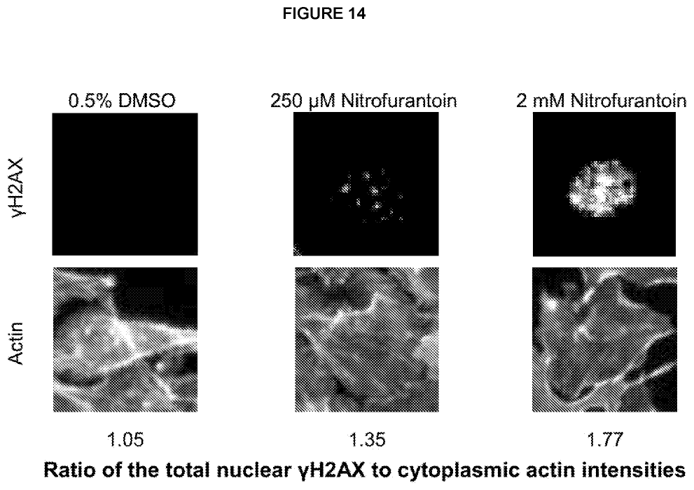

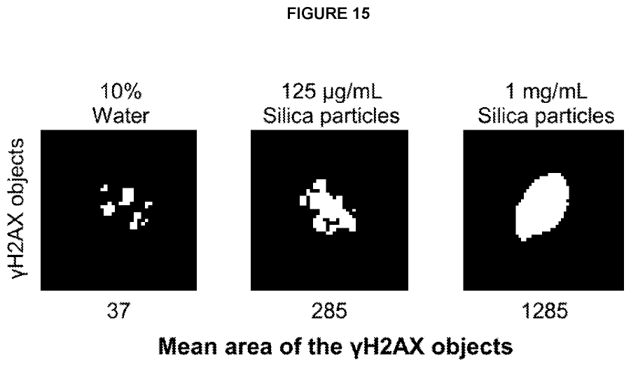

When the method of the invention was applied to predict the toxicity of compounds on BEC and AVC cells, similar results were obtained as for the kidney toxicity analysis. The results are four novel DNA, chromosomal and cytoskeletal features that can predict pulmonary toxicity of soluble or particulate compounds with test accuracy ranging from ˜86%-100% (Table 5). The best single features for A549 and BEAS-2B cells are different. For A549 cells, we found that the best single feature (f_{best}) for the soluble compounds was the mean entropy of the actin GLCM at the whole cell region (98.0% training and 86.2% test accuracies), and for the particulate compounds was the information measure of correlation 1 of the γH2AX stains at the nuclear region (100.0% training and 100.0% test accuracies) (FIG. 13a). For BEAS-2B cells, the best single feature (f_{best}) for the soluble compounds was the ratio of the total γH2AX to actin intensities at the nuclear to the cytoplasm region (99.1% training and 86.3% test accuracies) (FIGS. 13b and 14). There were 5 best single feature f_{best} for particulate compounds (100.0% training and 100.0% test accuracies) and all of them were related to either the γH2AX and/or actin stains, i.e. the total area of the γH2AX objects, ratio of the actin object intensities at the cytoplasm region, etc (FIGS. 13b and 15).

TABLE 5
Summary of pulmonotoxicity prediction performances for the best
single-feature classifiers.

| | | Com- | | Test | | |
|---|---|---|---|---|---|---|
| Cell | Com- | pound | | acc. | Sensi. | Speci. |
| lines | pound | number | Best feature name | (%) | (%) | (%) |
| A549 | Soluble | 39 | Mean entropy of the actin GLCM at | 86.2 | 83.8 | 88.8 |
| | | | the whole cell region | | | |
| | Particulate | 10 | Information measure of the correlation | 100.0 | 100.0 | 100.0 |
| | | | 1 of the γH2AX stains at the nuclear | | | |
| | | | region | | | |
| BEAS2B | Soluble | 39 | Ratio of the total nuclear γH2AX to | 86.3 | 87.7 | 84.6 |
| | | | cytoplasmic actin intensities | | | |
| | Particulate | 10 | Mean area of the γH2AX objects | 100.0 | 100.0 | 100.0 |
| | | | Ratio of the γH2AX object intensity at | | | |
| | | | the chromosomal region to all γH2AX | | | |
| | | | objects | | | |
| | | | Ratio of the actin object intensity at the | | | |
| | | | cytoplasm region to all actin objects | | | |
| | | | Ratio of the total γH2AX to DNA | | | |
| | | | intensities at the nuclear region | | | |
| | | | Mean angular second moment of the | | | |
| | | | γH2AX GLCM at the nuclear region | | | |

### Discussion

The current study shows that cell death of in vitro cultivated PTCs is induced to a variable degree by different PTC-toxic compounds (FIG. 10). This finding is in agreement with our and other previous results on predicting nephrotoxicity in humans (Wu et al., Toxicol In Vitro 23: 1170-1178 (2009); Li et al., Toxicol Res 2: 352-365 (2013)). The difficulties in using cell death as in vitro endpoint for predicting in vivo organ-specific toxicity may be related to the fact that in vivo compound-induced cell damage is not always associated with immediate cell death. For example, compound-induced PTC damage is often sub lethal, and associated with tubular dysfunction and chronic kidney disease instead of acute tubular necrosis (Kroshian et al., Am J Physiol 266: F21-30 (1994); Choudhury and Ahmed Nat Clin Pract Nephrol 2: 80-91 (2006)). The differences in the expression levels of xenobiotics-metabolizing enzymes and transporters may also play a role (Van der Hauwaert et al., Toxicol Appl Pharmacol 279: 409-418 (2014)). Generally, it remains a challenge to identify highly predictive endpoints for in vitro organ-specific toxicity models (Lin and Will Toxicol Sci 126: 114-127 (2012)). Specifically for kidney models, it has been consistently found that the use of general damage markers, such as ATP depletion; or potentially novel kidney-specific injury markers, such as kidney injury molecule-1 and neutrophil gelatinase-associated lipocalin, is of limited predictive value (Lin and Will Toxicol Sci 126: 114-127 (2012); Li et al., Toxicol Res 2: 352-365 (2013); Tiong et al., Mol Pharm 11: 1933-1948 (2014)). Therefore, the value of these current markers remains to be controversially discussed (Bonventre et al., Nat Biotechnol 28: 436-440 (2010); Vanmassenhove et al., Nephrol Dial Transplant 28: 254-273 (2013)).

A commonly used strategy to address such difficulties is to achieve an improved understanding of organ-specific injury mechanisms, and then select endpoints related to such mechanisms (Jennings et al., Arch Toxicol 88: 2099-2133 (2014)). However, this requires a priori knowledge of injury mechanisms, which may not be known for novel or not well-characterized compounds. In the current study, we took a more pragmatic approach for nephrotoxicity prediction without requiring a priori characterization of injury mechanisms, and directly searched for in vitro phenotypic features that could best predict in vivo toxicity. The results were six sets of nuclear and actin cytoskeletal features that could achieve ˜76-89% test accuracies in the kidney cells (Table 3); and ˜86-100% in the lung cells (Table 5). Our results show that, under in vitro conditions, most of the PTC-toxic, AVC- and BEC-toxic compounds induce similar phenotypic changes in the nucleus and actin cytoskeleton, even though these compounds may have dissimilar chemical structures.

The identification of features associated with specific cellular changes provides a mechanistic interpretation for our computational models. One of the most surprising findings of our study is that γH2AX, which is a known marker for genotoxicity and carcinogenesis (Mah et al., Leukemia 24: 679-686 (2010); Nikolova et al., Toxicol Sci 140: 103-117 (2014)), was also induced by many compounds with diverse chemical structures. Some of our reference compounds, such as cisplatin, 5-fluorouracil and aristolochic acid, are known to directly interfere with DNA replication and cause double strand breaks (Heidelberger et al. 1957; Lieberthal et al., Am J Physiol—Ren Physiol 270: F700-F708 (1996); Arlt Mutagenesis 17: 265-277 (2002)). However, most of our other reference PTC-toxic compounds are not known to interact with nucleic acids directly. Our observations are in agreement with other recent studies, which found that DNA damage responses were induced after renal ischemia-reperfusion injury in vivo and ATP-depletion injury in vitro (Ma et al., Biochim Biophys Acta BBA—Mol Basis Dis 1842: 1088-1096 (2014)), and also after treatments of angiotensin II, which is not known to interact with DNA, in isolated perfused mouse kidneys and PTC cultures in vitro (Schmid et al., Cancer Res 68: 9239-9246 (2008)). These observed DNA damage responses may be due to oxidative stress and/or oxidative DNA damage (Schmid et al., Cancer Res 68: 9239-9246 (2008); Ma et al., Biochim Biophys Acta BBA—Mol Basis Dis 1842: 1088-1096 (2014)). Some of our reference compounds, such as gentamicin, are known to induce oxidative stress and generate reactive-oxygen-species (ROS)-induced DNA damage (Quiros et al., Toxicol Sci 119: 245-256 (2011)). Irrespective of the underlying molecular mechanisms, our study show that in both primary PTCs and an immortalized PT cell line, γH2AX and DNA features were highly predictive of xenobiotics-induced PTC toxicity. Importantly, this also demonstrates how unexpected but common compound-induced cellular response and injury may be uncovered in an unbiased approach that does not focus on particular mechanisms.

Interestingly, the retention of cytoskeleton features in our final feature sets suggest that the DNA/γH2AX and actin markers provide complimentary and non-redundant information about cellular responses to PTC-toxic compounds. Remodeling of the actin cytoskeleton induced by various types of toxic compounds has been reported in PTCs (Elliget et al., Cell Biol Toxicol 7: 263-280 (1991); Kroshian et al., Am J Physiol 266: F21-30 (1994)). In addition to the cytoplasm, actin filaments are also localized in the nucleus, and actin-related proteins (Arps) are parts of chromatin remodeling complexes (Shen et al., Mol Cell 12: 147-155 (2003)). Therefore, the possible role of actin filaments in DNA damage responses will be an important question for future research.

There were two main factors that contributed to the high accuracy of our computational models. The first factor was the use of image-based phenotypic features, which allowed us to quantitatively measure changes in the spatial organizations of cells and subcellular organelles, such as DNA, histone modifications and actin cytoskeleton. We found that Haralick's texture features of the chromatin and cytoskeleton contained highly discriminative information, which would be lost under population-averaged or non-image-based measurements. Our results also show that the initial set of 129 general phenotypic features was a good starting point for screening predictive toxicity endpoints. The second factor that contributed to the high accuracy was the design our reference compounds and performance evaluation methodology. The inclusion of diverse compounds and non-PTC-toxic toxicants in the negative reference groups allowed us to search for more specific phenotypic features. We also ensured that training and test data were statistically independent from each other. For example, the feature normalization and elimination parameters were always determined using the training data only, but applied to both the training and test data in every single fold in our cross-validation procedure. In conclusion, our study demonstrates the feasibility of predicting the human nephrotoxicity of xenobiotics compounds with diverse chemical structures using high-throughput imaging, phenotypic profiling, and machine learning methods.

### REFERENCES

Abraham V C, Towne D L, Waring J F, et al (2008) Application of a High-Content Multiparameter Cytotoxicity Assay to Prioritize Compounds Based on Toxicity Potential in Humans. J Biomol Screen 13:527-537. doi: 10.1177/1087057108318428.
Arlt V M (2002) Aristolochic acid as a probable human cancer hazard in herbal remedies: a review. Mutagenesis 17:265-277. doi: 10.1093/mutage/17.4.265.
Bonventre J V, Vaidya V S, Schmouder R, et al (2010) Next-generation biomarkers for detecting kidney toxicity. Nat Biotechnol 28:436-440. doi: 10.1038/nbt0510-436.
Breiman L (2001) Random Forests. Mach Learn 45:5-32. doi: 10.1023/A:1010933404324.
Cherkasov A, Muratov E N, Fourches D, et al (2014) QSAR Modeling: Where Have You Been? Where Are You Going To? J Med Chem 57:4977-5010. doi: 10.1021/jm4004285.
Choudhury D, Ahmed Z (2006) Drug-associated renal dysfunction and injury. Nat Clin Pract Nephrol 2:80-91. doi: 10.1038/ncpneph0076.
Deptala A, Bedner E, Gorczyca W, Darzynkiewicz Z (1998) Activation of nuclear factor kappa B (NF-κB) assayed by laser scanning cytometry (LSC). Cytometry 33:376-382. doi: 10.1002/(SICI)1097-0320(19981101)33:3<376:AID-CYTO13>3.0.CO;2-Q.
Elliget K A, Phelps P C, Trump B F (1991) HgCl2-induced alteration of actin filaments in cultured primary rat proximal tubule epithelial cells labeled with fluorescein phalloidin. Cell Biol Toxicol 7:263-280.
Feng Y, Mitchison T J, Bender A, et al (2009) Multi-parameter phenotypic profiling: using cellular effects to characterize small-molecule compounds. Nat Rev Drug Discov 8:567-578. doi: 10.1038/nrd2876.
Haralick R M, Shanmugam K, Dinstein 1(1973) Textural Features for Image Classification. IEEE Trans Syst Man Cybern SMC-3:610-621. doi: 10.1109/TSMC.1973.4309314.
Heidelberger C, Chaudhuri N K, Danneberg P, et al (1957) Fluorinated Pyrimidines, A New Class of Tumour-Inhibitory Compounds. Publ Online 30 Mar. 1957 Doi101038179663a0 179:663-666. doi: 10.1038/179663a0.
Hoffmann D, Adler M, Vaidya V S, et al (2010) Performance of Novel Kidney Biomarkers in Preclinical Toxicity Studies. Toxicol Sci 116:8-22. doi: 10.1093/toxsci/kfq029.
Hong S J, Ghosh R N (2015) Predicting toxicity of a compound over a range of concentrations. U.S. patent application Ser. No. 14/334,453.
Jakob B, Splinter J, Conrad S, et al (2011) DNA double-strand breaks in heterochromatin elicit fast repair protein recruitment, histone H2AX phosphorylation and relocation to euchromatin. Nucleic Acids Res 39:6489-6499. doi: 10.1093/nar/gkr230.
Jang K-J, Mehr A P, Hamilton G A, et al (2013) Human kidney proximal tubule-on-a-chip for drug transport and nephrotoxicity assessment. Integr Biol 5:1119-1129. doi: 10.1039/C311340049B.
Jennings P, Schwarz M, Landesmann B, et al (2014) SEURAT-1 liver gold reference compounds: a mechanism-based review. Arch Toxicol 88:2099-2133. doi: 10.1007/s00204-014-1410-8.
Kandasamy K, Chuah J K C, Su R, et al (2015) Prediction of drug-induced nephrotoxicity and injury mechanisms with human induced pluripotent stem cell-derived cells and machine learning methods. Sci Rep. doi: 10.1038/srep12337.
Kellerman P S, Clark R A, Hoilien C A, et al (1990) Role of microfilaments in maintenance of proximal tubule structural and functional integrity. Am J Physiol 259:F279-285.
Krewski D, Jr D A, Andersen M, et al (2010) Toxicity Testing in the 21st Century: A Vision and a Strategy. J Toxicol Environ Health Part B 13:51-138. doi: 10.1080/10937404.2010.483176.
Kroshian V M, Sheridan A M, Lieberthal W (1994) Functional and cytoskeletal changes induced by sublethal injury in proximal tubular epithelial cells. Am J Physiol 266:F21-30.
Laksameethanasan D, Tan R, Toh G, Loo L-H (2013) cellXpress: a fast and user-friendly software platform for profiling cellular phenotypes. BMC Bioinformatics 14 Suppl 16:S4. doi: 10.1186/1471-2105-14-S16-S4.
Lieberthal W, Triaca V, Levine J (1996) Mechanisms of death induced by cisplatin in proximal tubular epithelial cells: apoptosis vs. necrosis. Am J Physiol—Ren Physiol 270:F700-F708.
Lilienblum W, Dekant W, Foth H, et al (2008) Alternative methods to safety studies in experimental animals: role in the risk assessment of chemicals under the new European Chemicals Legislation (REACH). Arch Toxicol 82:211-236. doi: 10.1007/s00204-008-0279-9.
Lin Z, Will Y (2012) Evaluation of Drugs With Specific Organ Toxicities in Organ-Specific Cell Lines. Toxicol Sci 126:114-127. doi: 10.1093/toxsci/kfr339.
Li Y, Kandasamy K, Chuah J K C, et al (2014) Identification of Nephrotoxic Compounds with Embryonic Stem-Cell-Derived Human Renal Proximal Tubular-Like Cells. Mol Pharm 11:1982-1990. doi: 10.1021/mp400637s.
Li Y, Oo Z Y, Chang S Y, et al (2013) An in vitro method for the prediction of renal proximal tubular toxicity in humans. Toxicol Res 2:352-365. doi: 10.1039/C3TX50042J.
Loo L-H, Wu L F, Altschuler S J (2007) Image-based multivariate profiling of drug responses from single cells. Nat Methods 4:445-453. doi: 10.1038/nmeth1032.
Mah L-J, El-Osta A, Karagiannis T C (2010) γH2AX: a sensitive molecular marker of DNA damage and repair. Leukemia 24:679-686. doi: 10.1038/leu.2010.6.
Matsusaka T, Fujikawa K, Nishio Y, et al (1993) Transcription factors NF-IL6 and NF-kappa B synergistically activate transcription of the inflammatory cytokines, interleukin 6 and interleukin 8. Proc Natl Acad Sci 90:10193-10197.
Ma Z, Wei Q, Dong G, et al (2014) DNA damage response in renal ischemia-reperfusion and ATP-depletion injury of renal tubular cells. Biochim Biophys Acta BBA—Mol Basis Dis 1842:1088-1096. doi: 10.1016/j.bbadis.2014.04.002.
Nikolova T, Dvorak M, Jung F, et al (2014) The H2AX Assay for Genotoxic and Nongenotoxic Agents: Comparison of H2AX Phosphorylation with Cell Death Response. Toxicol Sci 140:103-117. doi: 10.1093/toxsci/kfu066.
O'Brien P J, Irwin W, Diaz D, et al (2006) High concordance of drug-induced human hepatotoxicity with in vitro cytotoxicity measured in a novel cell-based model using high content screening. Arch Toxicol 80:580-604. doi: 10.1007/s00204-006-0091-3.
Paull T T, Rogakou E P, Yamazaki V, et al (2000) A critical role for histone H2AX in recruitment of repair factors to nuclear foci after DNA damage. Curr Biol 10:886-895. doi: 10.1016/S0960-9822(00)00610-2.
Quiros Y, Vicente-Vicente L, Morales A I, et al (2011) An Integrative Overview on the Mechanisms Underlying the Renal Tubular Cytotoxicity of Gentamicin. Toxicol Sci 119:245-256. doi: 10.1093/toxsci/kfq267.
Rogakou E P, Pilch D R, Orr A H, et al (1998) DNA Double-stranded Breaks Induce Histone H2AX Phosphorylation on Serine 139. J Biol Chem 273:5858-5868. doi: 10.1074/jbc.273.10.5858
Sawai H, Domae N (2011) Discrimination between primary necrosis and apoptosis by necrostatin-1 in Annexin V-positive/propidium iodide-negative cells. Biochem Biophys Res Commun 411:569-573. doi: 10.1016/j.bbrc.2011.06.186.
Sayes C M, Reed K L, Warheit. 2007. Assessing Toxicity of Fine and Nanoparticles: Comparing In Vitro Measurements to In Vivo Pulmonary Toxicity Profiles. Toxicol Sci 97(1):163-180.
Schmid U, Stopper H, Schweda F, et al (2008) Angiotensin II Induces DNA Damage in the Kidney. Cancer Res 68:9239-9246. doi: 10.1158/0008-5472.CAN-08-1310.
Seagrave J, McDonald J D, Mauderly J L. 2005. In vitro versus in vivo exposure to combustion emissions. Exp Toxicol Pathol 57:233-238.
Shen X, Ranallo R, Choi E, Wu C (2003) Involvement of Actin-Related Proteins in ATP-Dependent Chromatin Remodeling. Mol Cell 12:147-155. doi: 10.1016/S1097-2765(03)00264-8.
Sternberg S R (1983) Biomedical Image Processing. Computer 16:22-34. doi: 10.1109/MC.1983.1654163.
Su R, Li Y, Zink D, Loo L-H (2014) Supervised prediction of drug-induced nephrotoxicity based on interleukin-6 and -8 expression levels. BMC Bioinformatics 15:S16. doi: 10.1186/1471-2105-15-S16-S16.
Tiong H Y, Huang P, Xiong S, et al (2014) Drug-Induced Nephrotoxicity: Clinical Impact and Preclinical in Vitro Models. Mol Pharm 11:1933-1948. doi: 10.1021/mp400720w.
Tolosa L, Pinto S, Donato M T, et al (2012) Development of a Multiparametric Cell-based Protocol to Screen and Classify the Hepatotoxicity Potential of Drugs. Toxicol Sci 127:187-198. doi: 10.1093/toxsci/kfs083.
Torgerson W S (1952) Multidimensional scaling: I. Theory and method. Psychometrika 17:401-419. doi: 10.1007/BF02288916
Trevor Hastie, Robert Tibshirani, Jerome Friedman (2009) The Elements of Statistical Learning—Data Mining, Inference, and Prediction, 2nd edn. Springer
Van der Hauwaert C, Savary G, Buob D, et al (2014) Expression profiles of genes involved in xenobiotic metabolism and disposition in human renal tissues and renal cell models. Toxicol Appl Pharmacol 279:409-418. doi: 10.1016/j.taap.2014.07.007
Vanmassenhove J, Vanholder R, Nagler E, Van Biesen W (2013) Urinary and serum biomarkers for the diagnosis of acute kidney injury: an in-depth review of the literature. Nephrol Dial Transplant 28:254-273. doi: 10.1093/ndt/gfs380
Wu Y, Connors D, Barber L, et al (2009) Multiplexed assay panel of cytotoxicity in HK-2 cells for detection of renal proximal tubule injury potential of compounds. Toxicol In Vitro 23:1170-1178. doi: 10.1016/j.tiv.2009.06.003
Xu J J, Henstock P V, Dunn M C, et al (2008) Cellular Imaging Predictions of Clinical Drug-Induced Liver Injury. Toxicol Sci 105:97-105. doi: 10.1093/toxsci/kfn109

1.-37. (canceled)

38. An in vitro method of predicting whether a test compound will be toxic for a specific cell type in vivo, the method comprising:
(a) contacting at least one test population of cells with the test compound at a single concentration or over a range of concentrations,
(b) labeling and imaging the cells with one or more biomolecular markers,
(c) segmenting the cells and identifying whole-cell regions and one or more subcellular regions from the cells,
(d) determining if cell loss or death has occurred at the highest test concentrations; if so, stop and predict the compound is toxic,
(e) obtaining one or more quantified spatial-dependent and -independent phenotypic features in the test populations,
(f) obtaining multiple dose response curves (DRCs) from the features,
(g) obtaining quantified parameters of the DRCs wherein the DRC parameters are quantitated using the maximum response value Δₘₐₓ for each phenotypic feature from a DRC of the test compound, and
(h) comparing the quantitated DRC parameters to a reference set of quantitated DRC parameter data; said reference quantitated DRC parameter data being derived from two groups;
(i) compounds with known in vivo toxicity to the cell type, and (ii) compounds not known to be toxic to the cell type in vivo.

39. The method of claim 38, wherein said specific cell type is selected from the group comprising renal proximal tubular cells (PTCs), bronchial epithelial cells (BECs), and/or alveolar cells (AVCs).

40. The method of claim 38, wherein said one or more quantitated phenotypic features are associated with characteristics selected from the group comprising DNA damage response, actin filament integrity, whole cell morphology and cell count.

41. The method of claim 38, wherein said one or more phenotypic features are quantitated based on (i) one or more of the spatial-dependent features selected from the group comprising textural features, spatial correlation features, and ratios of markers at different subcellular regions; and (ii) one or more of the spatial-independent features selected from the group comprising intensity features, cell count, and morphology.

42. The method of claim 41, wherein said textural features include one or more of the statistics of the Haralick's grey-level co-occurrence matrix (GLCM) at specific sub- or whole-cellular regions, namely mean correlation of DNA GLCM at the nuclear region; mean entropy of DNA GLCM at the nuclear region; mean angular second moment of DNA GLCM at the nuclear region; standard deviation of the sum variance of DNA GLCM at the nuclear region;
mean sum entropy of actin GLCM at the whole cell region; mean entropy of actin GLCM at the whole cell region; standard deviation of the information measure of correlation 2 of γH2AX GLCM at the whole-cell region; and mean sum average of γH2AX GLCM at the whole cell region.

43. The method of claim 41, wherein said one or more of the spatial-dependent features comprises:
(a) a staining intensity feature selected from one or more of the group comprising normalized spatial correlation coefficient between DNA and actin intensities at the whole cell region; total actin intensity level at the inner cytoplasmic region; normalized spatial correlation coefficient between DNA and γH2AX intensities at the whole cell region; normalized spatial correlation coefficient between DNA and γH2AX intensities at the nuclear region and coefficient of variation of the DNA intensity at the nuclear region; or
(b) a staining intensity ratio feature selected from one or more of the group comprising ratio of the total γH2AX to DNA intensities at the whole cell region; the ratio of the total γH2AX to actin intensities at the nuclear region; and ratio of the total γH2AX intensity levels at the nuclear region to the whole cell region.

44. The method of claim 38, wherein the said one or more phenotypic features are selected from a group i) to iv) comprising:
i) mean sum entropy of the actin GLCM at the whole-cell region; coefficient of variation (CV) of the DNA intensity at the nuclear region; mean entropy of the actin GLCM at the whole-cell region; and mean angular second moment (ASM) of DNA GLCM at the nuclear region; or
ii) total actin intensity level at the inner cytoplasmic region; mean angular second moment (ASM) of DNA GLCM at the nuclear region; standard deviation of the information measure of correlation 2 of γH2AX GLCM at the whole-cell region; and cell count; or
iii) normalized spatial correlation coefficient between DNA and γH2AX intensities at the whole-cell region; normalized spatial correlation coefficient between DNA and actin intensities at the whole-cell region; mean sum average of γH2AX GLCM at the whole-cell region; ratio of the total γH2AX to DNA intensities at the whole-cell region; and standard deviation of the sum variance of DNA GLCM at the nuclear region; or
iv) mean entropy of the DNA GLCM at the nuclear region; ratio of the total γH2AX intensity levels at the nuclear region to the whole-cell region; mean correlation of actin GLCM; and mean correlation of DNA GLCM at the nuclear region.

45. The method of claim 38, wherein cell toxicity is predicted using random-forest algorithm.

46. The method of claim 38, wherein the at least one test population of cells are derived from somatic cells.

47. The method of claim 38, wherein said contacting is performed over a period of time of at least 1-48 hours; and/or
comprises adding the test compound to the at least one test population of cells at a concentration of about 1 μg/ml to about 1000 μg/ml.

48. The method of claim 38, wherein said imaging techniques comprise high-throughput microscopy image capture.

49. A computer-implemented method of predicting in vivo cell toxicity of a test compound using at least one test population of the cells subjected to the test compound in vitro, the method comprising:
(a) receiving, by a computer processor, an image of the test population of the cells;
(b) extracting, by the computer processor, one or more spatial-dependent phenotypic features associated with the test population of cells from the image, the one or more spatial-dependent phenotypic feature characterizing a spatial distribution of biomolecules associated with the cells;
(c) obtaining one or more quantitated dose response curve (DRC) parameters describing the DRC of the respective one or more spatial-dependent phenotypic features, wherein the quantitated DRC parameter is obtained using the maximum response value Δₘₐₓ; and
(d) inputting said one or more quantitated DRC parameters to a predictive model to generate a prediction of in vivo cell toxicity of the test compound.

50. A method according to claim 49, wherein the cells are renal proximal tubular cells (PTCs), bronchial epithelial cells (BECs), or alveolar cells (AVCs).

51. A method according to claim 49, wherein said image comprises a plurality of images each representing the test population of cells imaged using a respective imaging channel emphasizing a type of biomolecules associated with the cells.

52. A method according to claim 51, wherein each of the plurality of images represents a distribution of a type of biomarkers targeting the corresponding type of biomolecules.

53. A method according to claim 49, wherein operation (b) comprises segmenting the cells using the image, and extracting the one or more spatial-dependent phenotypic features using intensity values of the image corresponding to the segmented cells.

54. A method according to claim 49, wherein the one or more spatial-dependent phenotypic features are selected from the group comprising features characterizing DNA structure alterations, chromatin structure alterations and Actin filament structure alterations of the cells.

55. A method according to claim 49, wherein the predicative model is obtained using a supervised learning algorithm trained with a set of training data.

56. A method according to claim 55, wherein said set of training data comprises a plurality of candidate quantitated spatial-dependent dose response curve (DRC) parameters characterizing a corresponding plurality of spatial-dependent phenotypic features associated with control populations of cells; said control populations of cells having been respectively subjected to: (i) compounds known to be toxic to the cells in vivo; (ii) compounds not known to be toxic to the cells in vivo.

57. A method according to claim 49, further comprising extracting one or more spatial-independent phenotypic features associated with the at least one test population of cells, and obtaining the one or more quantitated dose response curve (DRC) parameters further using the one or more spatial-independent phenotypic features.

58. A method according to claim 49, wherein operation (c) comprises obtaining the quantitated DRC parameter at a pre-defined concentration of the test compound.
